(19) United States
(12) Patent Application Publication
(10) Pub. No.: US 2012/0329865 A1
(43) Pub. Date: Dec. 27, 2012

(54) MOLECULES RELATED hERG ION CHANNELS AND THE USE THEREOF

(76) Inventors: Huayun Deng, Painted Post, NY (US); Ye Fang, Painted Post, NY (US); Ann MeeJin Ferrie, Painted Post, NY (US); Mingqian He, Horseheads, NY (US); Weijun Niu, Painted Post, NY (US); Haiyan Sun, Chandler, AZ (US); Elizabeth Tran, Painted Post, NY (US); Ying Wei, Painted Post, NY (US)

(21) Appl. No.: 13/519,424

(22) PCT Filed: Dec. 16, 2010
(86) PCT No.: PCT/US2010/060655

§ 371 Date: Jun. 27, 2012

Related U.S. Application Data
(60) Provisional application No. 61/291,742, filed on Dec. 31, 2009.

(51) Int. Cl. A61K 31/341; C12N 5/071; A61P 35/00; G01N 21/17; A61K 31/343; A61P 35/02

(52) U.S. Cl. 514/462; 514/471; 435/29; 435/375

(57) ABSTRACT

Disclosed are compounds having structural formula (I, II) or a pharmaceutically acceptable sale, solvate, clathrate, or prodrug thereof, wherein R¹, R², R³, R⁶, R⁵, and R⁴ are defined herein. These compounds can be useful as therapeutic agents for modulating hERG ion channels, and for improving prevention and treatment of hERG associated cardiac repolarization disorders.

### CROSS-REFERENCE TO RELATED APPLICATIONS

This application claims the benefit of priority under 35 U.S.C. §119(e) of U.S. Provisional Application Ser. No. 61/291,742 filed on Dec. 31, 2009.

### BACKGROUND

The human ether-à-go-go related gene (hERG) encodes the pore-forming a subunit of a voltage gated potassium channel (Kv11.1). hERG channel are expressed in various tissues including cardiac myocytes, neurons, pancreatic β cells, smooth muscles and some cancer cells.

hERG current is best known as the major component of the delayed rectifier current Iₖᵣ in the heart which is important for the action potential repolarization. Genetic mutations in hERG channel have been known to cause the inherited long QT syndrome (LQTS); a disease may result in patient sudden death. Drugs that can block hERG current, or inhibit hERG channel protein trafficking may cause the acquired LQTS. To minimize the drug induced cardiac risk, all compounds under consideration for Investigational New Drug (IND) applications need to be tested for hERG interaction in compliance with GLP principles according to the ICH S7A and ICH S7B guidelines.

Besides playing a critical role in cardiac myocytes, increasing evidence has shown that hERG channel expression level was elevated in several types of cancer cells including leukemia, colon cancer, gastric cancer, breast cancer and lung cancer cells. It is not clear why the hERG channel is overexpressed in cancer cells, but it is indicated that hERG channel may play a role in cancer cell proliferation.

hERG channel function is modulated by protein kinase A and protein kinase C involved pathways. hERG current is acutely inhibited when hERG protein is phosphorylated by the activation of cAMP dependent PKA. Elevated level of cAMP and prolonged PKA activity can also increase the hERG protein expression. hERG current may also be modulated adrenergic receptors through PKA and PKC.

hERG channel has unique pore region that can accommodate structure diverse channel blockers. A comparatively large inner cavity and the presence of particular aromatic amino acid residues (Y652 and F656) on the inner (S6) helices of the channel are important features that allow hERG to accommodate and bind disparate drugs.

The most commonly used class III antiarrhymics agents affected multiple targets, particularly as antiadrenergics; for example, amiodarone has pharmacokinetic drug-drug interactions and many potential side effects. However, hERG blockade has been used as an antiarrhythmic drug action used for prophylaxis of re-entrant arrhythmias. Unlike other class III antiarrhythmics such as amiodarone and racemic sotalol, the FDA approved and high affinity hERG blocker dofetilide has pure class III activity via hERG blockade. Several other hERG-blocking class III antiarrhythmics have been developed, such as ibutilide and clofilium, although clofilium is not used in clinical practice. There are several mechanisms accounting for the hERG blockage. All of these clinical drugs act by blocking the channel at the canonical hERG drug binding site inside the pore cavity, with block being dependent upon the channel gating before the drug can reach its target binding site (open- or inactivated-state blockade). However, there are other state-dependent mechanisms of hERG blockade by drugs (e.g., closed-state-dependent and mixed-state-dependent). Different examples of hERG blockade can often be distinguished by their site of action—some hERG blockers do not always act strictly via the canonical hERG drug-binding site involving the aromatic residues in S6 inside the pore cavity and the class III antiarrhythmic dronedarone has also been shown not to act precisely there, despite its open/inactivated-state dependence. It has been suggested that the different types of hERG blockade (i.e., different state-dependences or sites of action) may be associated with different levels of arrhythmogenic risk and that simply measuring the hERG IC50 value is not always sufficient for understanding the ‘true’ hERG liability (the arrhythmogenic risk) associated with a particular hERG-blocking drug.

In addition to the various hERG channel blockers, seven hERG channel activators have been identified, including RPR260243, NS1643, NS3623, PD-118057, PD-307243, mallotoxin and A-935142 (see Su, Z., et al. Electrophysiologic characterization of a novel hERG channel activator. Biochem Pharm 77:1383, 2009). These hERG activators have diverse chemical structures and enhance the hERG channel activity by different mechanisms. Among these known hERG activators, PD-118057, NS3623 and RPR260243 have been shown to shorten both the ventricular AP duration and the QT interval. RPR260243 and PD-118057 can reverse the AP prolonging effects of dofetilide. The mechanism of action of these channel activators is varied. NS1643 and NS3623 primarily reduce the inactivation of hERG by shifting its voltage dependence rightward; neither compound was designed to interact with the S5-pore linker, and their sites of action with the hERG channel are as yet unknown. Mallotoxin affects all three, strongly shifting the activation curve leftward, but also slowing deactivation and having minor effects on inactivation. In addition, it may be possible to modulate hERG activity with drugs acting on protein kinases, as hERG current can be modulated by protein kinase A and protein kinase C activity. The discovery of these structurally diverse hERG activators could be an immense breakthrough in terms of treating clinical conditions with hERG targets, as well as potentially increasing the safety of other drugs known to block hERG.

### SUMMARY

Disclosed are compositions which modulate hERG.

Disclosed are compounds having structural formula (I, II) or a pharmaceutically acceptable sale, solvate, clathrate, or prodrug thereof, wherein R¹, R², R³, R⁶, R⁵, and R⁴ are defined herein.

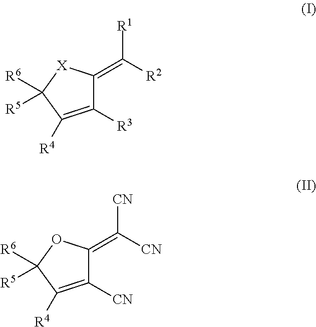

These compounds can be useful as therapeutic agents for modulating hERG ion channels, and for improving prevention and treatment of hERG associated cardiac repolarization disorders.

Also disclosed are hERG pathway activators which are capable of activating hERG in a label free assay, but which do not cause any significant alteration in ion flux in an ion flux assay. These hERG pathway activators can be used in label free assays and for testing and identifying hERG activators, as well as in assays related to toxicity assays.

Disclosed herein, the hERG activators can be used to override the LQTS inducing drugs and thus to improve the therapeutic potentials and safety profiles of existing and new drugs. These hERG activators can remove bound hERG blocker drug molecules in hERG ion channels, thus reducing any potential liability of these drug molecules acting via hERG channels.

Also disclosed, this Structure-Activity-Relation functionality of the class hERG activators can be used to design new generations of anti-cancer drugs having desired cross reactivity profiles with hERG minimizing potential LQTS. hERG channels are known to be expressed in some cancers, and also play a proliferative role in the growth of these cancers. Thus a hERG blocker that blocks the hERG-mediated signaling but without any significant impact on hERG current could be beneficial to preventing or suppressing cancer development.

### BRIEF DESCRIPTION OF FIGURES

FIG. 1A-1D shows label-free optical biosensor DMR profiles of a representative hERG activator. (A) The DMR signal of compound E in the colon cancerous cell line HT29; (B) The DMR signal of the compound E in the hERG stably expressing engineered HEK293 cell line (HEK-hERG); (C) The DMR signal of the compound E in native HEK293 cells; (D) The modulation index of the compound E against the mallotoxin DMR signals in both HT29 and HEK-hERG cell lines. The compound E was assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 2A-2D shows label-free optical biosensor DMR profiles of a representative hERG activator. (A) The DMR signal of compound D in the colon cancerous cell line HT29; (B) The DMR signal of the compound Din the hERG stably expressing engineered HEK293 cell line (HEK-hERG); (C) The DMR signal of the compound Din native HEK293 cells; (D) The modulation index of the compound D against the mallotoxin DMR signals in both HT29 and HEK-hERG cell line. The compound Dwas assayed at 10 micromolar in all cells, while mallotoxin was at 16 micromolar. In graphs A, B and C, the respective net-zero DMR signals of cells in response to the vehicle (i.e., buffer) only were included as negative controls.

FIG. 3 shows Rb⁺ flux measurements of a representative hERG activator compound E and D using HEK-hERG cells under 5 mM KCl, in comparison with the known hERG activator mallotoxin as well as the known hERG blocker dofetilide. The modulators were assayed at either 10 micmolar or 50 micromolar, when KCl was maintained at 5 mM.

FIG. 4 shows that compound E, D and U (all at 25 micromolar) did not exhibit cytotoxicity on cancer cell line HT29 under in vitro culture condition. At least 4 replicates were used to calculate the averaged responses.

FIG. 5A-5E shows profiles of a representative hERG activator flufenamic acid. (A) The DMR signal of the anti-inflammatory drug flufenamic acid in the colon cancer cell line HT29; (B) The DMR signal of flufenamic acid in the engineered HEK293 cell line (HEK-hERG) stably expressing hERG; (C) The DMR signal of flufenamic acid in native HEK293 cells; (D) The modulation index of flufenamic acid against the mallotoxin DMR signals in both HT29 and HEK-hERG cell lines. Flufenamic acid was assayed at 10 micromolar in all cells, while mallotoxin was at 16 micromolar. In graphs A, B and C, the respective net-zero DMR signals of cells in response to the vehicle (i.e., buffer) only were included as negative controls. (E) The electrophysiological diagrams showing the effect of 50 micromolar flufenamic acid on the current of the HEK-hERG cells. The gray curves represented the electrophysiological recording of HEK-hERG cells before the addition of flufenamic acid, while the black curves showed the electrophysiological recording of the same cell after the addition of flufenamic acid. (a, b, c) indicated the three phases of hERG current measurements as defined herein.

FIG. 6A-6E shows profiles of a representative hERG pathway activator diflunisal. Diflunisal is also a known prostaglandin synthetase inhibitor. (A) The DMR signal of diflunisal in the colon cancer cell line HT29; (B) The DMR signal of diflunisal in the engineered HEK293 cell line (HEK-hERG) stably expressing hERG; (C) The DMR signal of diflunisal in native HEK293 cells; (D) The modulation index of diflunisal against the mallotoxin DMR signals in both HT29 and HEK-hERG cell lines. Diflunisal was assayed at 10 micromolar in all cells, while mallotoxin was at 16 micromolar. In graphs A, B and C, the respective net-zero DMR signals of cells in response to the vehicle (i.e., buffer) only were included as negative controls. (E) The electrophysiological diagrams showing the effect of 50 micromolar diflunisal on the current of the HEK-hERG cells. The gray curves represented the electrophysiological recording of HEK-hERG cells before the addition of diflunisal, while the black curves showed the electrophysiological recording of the same cell after the addition of diflunisal.

FIG. 7A-7E shows profiles of a representative hERG activator B. (A) The DMR signal of B in the colon cancer cell line HT29; (B) The DMR signal of B in the engineered HEK293 cell line (HEK-hERG) stably expressing hERG; (C) The DMR signal of B in native HEK293 cells; (D) The modulation index of B against the mallotoxin DMR signals in both HT29 and HEK-hERG cell lines. B was assayed at 10 micromolar in all cells, while mallotoxin was at 16 micromolar. In graphs A, B and C, the respective net-zero DMR signals of cells in response to the vehicle (i.e., buffer) only were included as negative controls. (E) The electrophysiological diagrams showing the effect of 50 micromolar B on the current of the HEK-hERG cells. The gray curves represented the electrophysiological recording of HEK-hERG cells before the addition of B, while the black curves showed the electrophysiological recording of the same cell after the addition of B.

FIG. 8A-8H shows electrophysiological profiles of a series of hERG pathway activators. (A) to (H) A, C, D, F, H, I, J, and U. The electrophysiological diagrams showing the effect of these compounds on the current of the HEK-hERG cells. The gray curves represented the electrophysiological recording of HEK-hERG cells before the addition of a compound, while the black curves showed the electrophysiological recording of the same cell after the addition of the compound. (b) indicates the phase reflecting the tail current of hERG current. All compounds were assayed at 50 micromolar.

FIG. 9 shows electrophysiological profiles of a well known hERG blocker dofeltilide. The electrophysiological diagrams showing the effect of these dofeltilide at 100 nM on the current of the HEK-hERG cells. The gray 910 curves represented the electrophysiological recording of HEK-hERG cells before the addition of a compound, while the black 920 curves showed the electrophysiological recording of the same cell after the addition of the compound. (b) indicates the phase reflecting the tail current of hERG current.

### DETAILED DESCRIPTION

hERG ion channel is a large tetramer protein. Depending on cellular backgrounds it may be complexed with other proteins. Therefore the cell background can affect assays looking at hERG modulation. The disclosed assays use three types of cells and cell lines: native hERG expressing cell line, a native cell line which does not express hERG, and an engineered cell line, that is engineered to express hERG. Since label-free biosensor cellular assays rely on a generic readout, such as DMR signal using optical biosensor or impedance signal using electric biosensor, and the biosensor signal often contains systems cell biology information of a target of interest (e.g., hERG channel), there can be a high percentage of false positives that could be a result from screening using a single hERG expressing cell. Combining three types of cells for detecting hERG modulation using a label-free biosensor can not only significantly reduce false positives, but also can increase the quality of potential hERG modulators identified. Furthermore, by using the three cell lines, a high resolution assessment picture of hERG specific screening modulators is created. The disclosed assays also use a hERG activator, such as mallotoxin, to generate the modulation index of a molecule against the hERG activator induced DMR signals in both hERG expressing cell lines. Such modulation indexes can be used further classifying the mode of actions of molecules acting on hERG channel or hERG channel signaling complexes.

Other proteins, such as other ion channels, such as the toll receptor, can be screened and characterized in similar ways, with three different cell lines and known modulators.

In certain label free cell assay methods, one has a cell line, a target, an activator (or modulator), and then a marker. These combinations can be used to assay for other modulators (See for example, WO2006108183 Fang, Y., et al. “Label-free biosensors and cells”).

The typical label free cell assay target approaches have high false positives.

The pathway label free cell assay tests get much information about the pathways and targets involved in these, but some specificity is lost at the target level. The methods disclosed herein, use the information that can be gained from label free target assays, and label free pathway assays, to arrive at a highly specific target assay.

The disclosed methods provide a higher resolution of information at a specific target then in previous label free integrated pharmacology methods, such as those disclosed in U.S. Ser. No. 12/623,708, Fang, Y., et al. “Methods of creating an index”. And U.S. Ser. No. 12/623,693, Fang, Y., et al., “Methods for characterizing molecules”. In the methods disclosed in U.S. Ser. No. 12/623,693, Fang, Y., et al., “Methods for characterizing molecules”, a panel of markers is chosen and assayed, and this information provides information about the pathways in the cell connected to the markers. The disclosed methods use identified cells, based on appropriate pathways for specific targets. In certain embodiments, the information used from methods disclosed in U.S. Ser. No. 12/623,693, Fang, Y., et al., “Methods for characterizing molecules”, can be used to provide the information and identified cells which can be used in the methods disclosed herein.

Disclosed are molecules which have a heretofor unknown activity, particularly on hERG channel. Disclosed are over 3000 compounds, which have been tested in a hERG ion channel label free biosensor cellular assay. These compounds include BioMol 640 FDA approved drug library, BioMol 80 Kinase Inhibitor Library, BioMol ActiCom library, Corning Internal Reference Compound Library, and Corning Internal Compound Library. According to the disclosed methods, a subset of these compounds are identified as hERG modulators, which can classified into three classes: a hERG activator, a hERG inhibitor, and a hERG signaling activator that is capable of activating hERG signaling but with or without impact on hERG current.

The traditional hERG ion channel assay involves assaying ion flux such as Rb+ flux using ion absorption assays, or assaying hERG currents directly using patch clamping methods. Traditionally, a molecule which causes increase in Rb+ flux and/or hERG currents is referred to a hERG activator, while a molecule which inhibits Rb+ flux and/or hERG currents is referred to a hERG inhibitor. The disclosed methods have identified different classes of hERG activators, including hERG ion channel activators and hERG pathway activators. These hERG activators may or may not result in detectable biosensor signals in cells, using label-free biosensor cellular assays. A hERG activator that results in a detectable biosensor signal in a hERG expressing cell via hERG or hERG signaling complex is also referred to a label-free biosensor hERG activator. A hERG pathway activator is a molecule which cause cell signaling mediated via hERG or hERG-associated signaling complex in cells. These hERG pathway activators are also referred to hERG signaling activators. A hERG pathway activator can be a classical a hERG activator, or a hERG inhibitor, based on its ability to potentiate or inhibit hERG ion flux and/or hERG current, respectively. The data herein discloses that there is a cell signaling activity of hERG, which can be dependent or independent on ion channel flux activity via hERG channels, heretofor unknown. A hERG pathway activator could lead to activation of specific pathway(s) downstream hERG channel directly, or hERG channel-associated signaling complex, thus triggering a detectable biosensor signal in cells. These pathways can include protein kinase A (PKA), protein kinase C (PKC), MAP kinase (MAPK) pathway, or integrin pathway, or any combinations of these pathways.

One clear indication from the present data is that prodrugs and drugs could effect hERG channels differently, as a traditional ion flux activator and as hERG pathway activator respectively.

Mallotoxin is commercially available, and it is a label free biosensor hERG activator and it is a hERG ion channel activator.

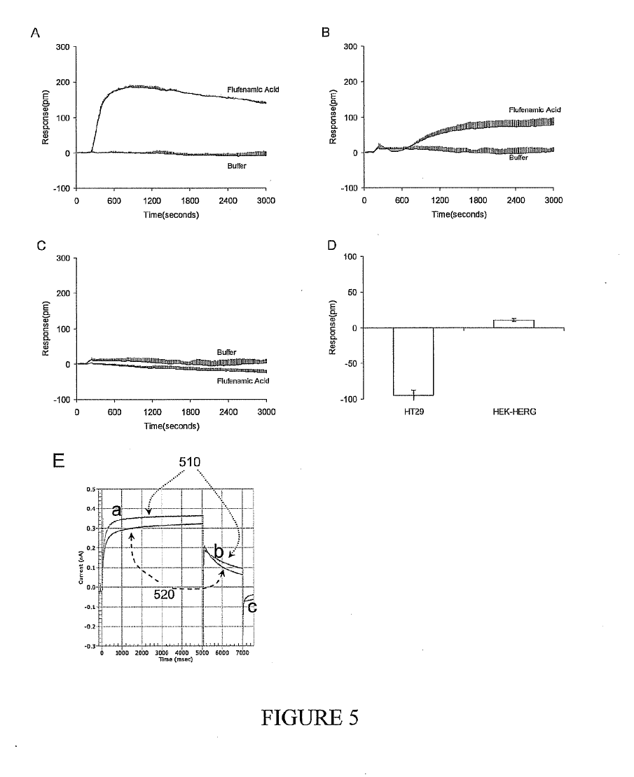

Also identified, flufenamic acid is a hERG pathway activator, a label free biosensor hERG activator and is a weak hERG ion flux activator, and a weak hERG current activator (FIG. 5).

Also identified, RPR260243, NS1643, NS3623, PD-118057, PD-307243, A-935142, niflumic acid, and diflunisal are label-free biosensor hERG activators.

Disclosed are hERG modulators, hERG activator, label-free biosensor hERG activator, hERG pathway activator, hERG ion channel activator, hERG inhibitor, hERG pathway inhibitor, and hERG ion channel inhibitor. These classes and specific examples of each can be used, for example, in the methods disclosed herein.

The methods disclosed herein, as well as the compositions and compounds which can be used in the methods, can arise from a number of different classes, such as materials, substance, molecules, and ligands. Also disclosed is a specific subset of these classes, unique to label free biosensor assays, called markers, for example, mallotoxin as a marker for hERG activation.

It is understood that mixtures of these classes, such as a molecule mixture are also disclosed and can be used in the disclosed methods.

In certain methods, unknown molecules, test molecules, drug candidate molecules as well as known molecules can be used.

In certain methods or situations, modulating or modulators play a role. Likewise, known modulators can be used.

In certain methods, as well as compositions, cells are involved, and cells can undergo culturing and cell cultures can be used as discussed herein.

The methods disclosed herein involve assays that use biosensors. In certain assays, they are performed in either an agonism or antagonism mode. Often the assays involve treating cells with one or more classes, such as a material, a substance, or a molecule. It is also understood that subjects can be treated as well, as discuss herein.

In certain methods, contacting between a molecule, for example, and a cell can take place. In the disclosed methods, responses, such as cellular response, which can manifest as a biosensor response, such as a DMR response, can be detected. These and other responses can be assayed. In certain methods the signals from a biosensor can be robust biosensor signals or robust DMR signals.

The disclosed methods utilizing label free biosensors can produce profiles, such as primary profiles, secondary profiles, and modulation profiles. These profiles and others can be used for making determinations about molecules, for example, and can be used with any of the classes discussed herein.

Also disclosed are libraries and panels of compounds or compositions, such as molecules, cells, materials, or substances disclosed herein. Also disclosed are specific panels, such as marker panels and cell panels.

The disclosed methods can utilize a variety of aspects, such as biosensor signals, DMR signals, normalizing, controls, positive controls, modulation comparisons, Indexes, Biosensor Indexes, DMR indexes, Molecule biosensor indexes, molecule DMR indexes, molecule indexes, modulator biosensor indexes, modulator DMR indexes, molecule modulation indexes, known modulator biosensor indexes, known modulator DMR indexes, marker biosensor indexes, marker DMR indexes, modulating the biosensor signal of a marker, modulating the DMR signal, potentiating, and similarity of indexes.

Any of the compositions, compounds, or anything else disclosed herein can be characterized in any way disclosed herein.

Disclosed are methods that rely on characterizations, such as higher and inhibit and like words.

In certain methods, receptors or cellular targets are used. Certain methods can provide information about signaling pathway(s) as well as molecule-treated cells and other cellular processes.

In certain embodiments, a certain potency or efficacy becomes a characteristic, and the direct action (of a drug candidate molecule, for example) can be assayed.

The disclosed methods can be performed on or with samples.

### A. DEFINITIONS

Various embodiments of the disclosure will be described in detail with reference to drawings, if any. Reference to various embodiments does not limit the scope of the disclosure, which is limited only by the scope of the claims attached hereto. Additionally, any examples set forth in this specification are not intended to be limiting and merely set forth some of the many possible embodiments for the claimed invention.

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Assaying

Assaying, assay, or like terms refers to an analysis to determine a characteristic of a substance, such as a molecule or a cell, such as for example, the presence, absence, quantity, extent, kinetics, dynamics, or type of an a cell's optical or bioimpedance response upon stimulation with one or more exogenous stimuli, such as a ligand or marker. Producing a biosensor signal of a cell's response to a stimulus can be an assay.

5. Assaying the Response

“Assaying the response” or like terms means using a means to characterize the response. For example, if a molecule is brought into contact with a cell, a biosensor can be used to assay the response of the cell upon exposure to the molecule.

6. Agonism and Antagonism Mode

The agonism mode or like terms is the assay wherein the cells are exposed to a molecule to determine the ability of the molecule to trigger biosensor signals such as DMR signals, while the antagonism mode is the assay wherein the cells are exposed to a maker in the presence of a molecule to determine the ability of the molecule to modulate the biosensor signal of cells responding to the marker.

7. Biosensor

Biosensor or like terms refer to a device for the detection of an analyte that combines a biological component with a physicochemical detector component. The biosensor typically consists of three parts: a biological component or element (such as tissue, microorganism, pathogen, cells, or combinations thereof), a detector element (works in a physicochemical way such as optical, piezoelectric, electrochemical, thermometric, or magnetic), and a transducer associated with both components. The biological component or element can be, for example, a living cell, a pathogen, or combinations thereof. In embodiments, an optical biosensor can comprise an optical transducer for converting a molecular recognition or molecular stimulation event in a living cell, a pathogen, or combinations thereof into a quantifiable signal.

8. Biosensor Response

A “biosensor response”, “biosensor output signal”, “biosensor signal” or like terms is any reaction of a sensor system having a cell to a cellular response. A biosensor converts a cellular response to a quantifiable sensor response. A biosensor response is an optical response upon stimulation as measured by an optical biosensor such as RWG or SPR or it is a bioimpedence response of the cells upon stimulation as measured by an electric biosensor. Since a biosensor response is directly associated with the cellular response upon stimulation, the biosensor response and the cellular response can be used interchangeably, in embodiments of disclosure.

9. Biosensor Signal

A “biosensor signal” or like terms refers to the signal of cells measured with a biosensor that is produced by the response of a cell upon stimulation.

10. Cell

Cell or like term refers to a small usually microscopic mass of protoplasm bounded externally by a semipermeable membrane, optionally including one or more nuclei and various other organelles, capable alone or interacting with other like masses of performing all the fundamental functions of life, and forming the smallest structural unit of living matter capable of functioning independently including synthetic cell constructs, cell model systems, and like artificial cellular systems.

A cell can include different cell types, such as a cell associated with a specific disease, a type of cell from a specific origin, a type of cell associated with a specific target, or a type of cell associated with a specific physiological function. A cell can also be a native cell, an engineered cell, a transformed cell, an immortalized cell, a primary cell, an embryonic stem cell, an adult stem cell, a cancer stem cell, or a stem cell derived cell.

Human consists of about 210 known distinct cell types. The numbers of types of cells can almost unlimited, considering how the cells are prepared (e.g., engineered, transformed, immortalized, or freshly isolated from a human body) and where the cells are obtained (e.g., human bodies of different ages or different disease stages, etc).

11. Cell Culture

“Cell culture” or “cell culturing” refers to the process by which either prokaryotic or eukaryotic cells are grown under controlled conditions. “Cell culture” not only refers to the culturing of cells derived from multicellular eukaryotes, especially animal cells, but also the culturing of complex tissues and organs.

12. Cell Panel

A “cell panel” or like terms is a panel which comprises at least two types of cells. The cells can be of any type or combination disclosed herein.

13. Cellular Response

A “cellular response” or like terms is any reaction by the cell to a stimulation.

14. Cellular Process

A cellular process or like terms is a process that takes place in or by a cell. Examples of cellular process include, but not limited to, proliferation, apoptosis, necrosis, differentiation, cell signal transduction, polarity change, migration, or transformation.

15. Cellular Target

A “cellular target” or like terms is a biopolymer such as a protein or nucleic acid whose activity can be modified by an external stimulus. Cellular targets are most commonly proteins such as enzymes, kinases, ion channels, and receptors.

16. Characterizing

Characterizing or like terms refers to gathering information about any property of a substance, such as a ligand, molecule, marker, or cell, such as obtaining a profile for the ligand, molecule, marker, or cell.

17. Comprise

Throughout the description and claims of this specification, the word “comprise” and variations of the word, such as “comprising” and “comprises,” means “including but not limited to,” and is not intended to exclude, for example, other additives, components, integers or steps.

18. Consisting Essentially of

“Consisting essentially of” in embodiments refers, for example, to a surface composition, a method of making or using a surface composition, formulation, or composition on the surface of the biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agents, a particular cell or cell line, a particular surface modifier or condition, a particular ligand candidate, or like structure, material, or process variable selected. Items that may materially affect the basic properties of the components or steps of the disclosure or may impart undesirable characteristics to the present disclosure include, for example, decreased affinity of the cell for the biosensor surface, aberrant affinity of a stimulus for a cell surface receptor or for an intracellular receptor, anomalous or contrary cell activity in response to a ligand candidate or like stimulus, and like characteristics.

19. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these molecules may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

20. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e., such that they are in proximity to mix or touch. For example, having a solution of composition A and cultured cell B and pouring solution of composition A over cultured cell B would be bringing solution of composition A in contact with cell culture B. Contacting a cell with a ligand would be bringing a ligand to the cell to ensure the cell have access to the ligand.

It is understood that anything disclosed herein can be brought into contact with anything else. For example, a cell can be brought into contact with a marker or a molecule, a biosensor, and so forth.

21. Compounds and Compositions

Compounds and compositions have their standard meaning in the art. It is understood that wherever, a particular designation, such as a molecule, substance, marker, cell, or reagent compositions comprising, consisting of, and consisting essentially of these designations are disclosed. Thus, where the particular designation marker is used, it is understood that also disclosed would be compositions comprising that marker, consisting of that marker, or consisting essentially of that marker. Where appropriate wherever a particular designation is made, it is understood that the compound of that designation is also disclosed. For example, if particular biological material, such as EGF, is disclosed EGF in its compound form is also disclosed.

22. Control

The terms control or “control levels” or “control cells” or like terms are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. For example, a control can refer to the results from an experiment in which the subjects or objects or reagents etc are treated as in a parallel experiment except for omission of the procedure or agent or variable etc under test and which is used as a standard of comparison in judging experimental effects. Thus, the control can be used to determine the effects related to the procedure or agent or variable etc. For example, if the effect of a test molecule on a cell was in question, one could a) simply record the characteristics of the cell in the presence of the molecule, b) perform a and then also record the effects of adding a control molecule with a known activity or lack of activity, or a control composition (e.g., the assay buffer solution (the vehicle)) and then compare effects of the test molecule to the control. In certain circumstances once a control is performed the control can be used as a standard, in which the control experiment does not have to be performed again and in other circumstances the control experiment should be run in parallel each time a comparison will be made.

23. Chemistry Terms

a) Alkyl

The term “alkyl” as used herein is a branched or unbranched saturated hydrocarbon moiety. “Unbranched” or “Branched” alkyls comprise a non-cyclic, saturated, straight or branched chain hydrocarbon moiety having from 1 to 24 carbons, 1 to 20 carbons, 1 to 15 carbons, 1 to 12 carbons, 1 to 8 carbons, 1 to 6 carbons, or 1 to 4 carbon atoms. It is understood that the term “alkyl” also encompass straight or branched chain hydrocarbon moiety having 1, 2, 3, 4, 5, 6, 7, 8, 9, 10, 11, 12, 13, 14, 15, 16, 17, 18, 19, 20, 21, 22, 23, or 24 carbon atoms. Examples of such alkyl radicals include methyl, ethyl, propyl, butyl, pentyl, hexyl, heptyl, octyl, n-propyl, iso-propyl, butyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to H, alkyl, alkenyl, alkynyl, hydroxyl, cycloalkyl, heterocyclyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituted of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

b) Alkenyl

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

c) Alkynyl

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butyryl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

d) Cycloalkyl

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

e) Cycloalkenyl

The term “cycloalkenyl” as used herein is a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

f) Alkoxy

The term “alkoxy” as used herein is an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like

g) Amino

The term “amino” as used herein is a moiety comprising a N radical substituted with zero, one or two organic substituent groups, which include but are not limited to alkyls, substituted alkyls, cycloalkyls, aryls, or arylalkyls. If there are two substituent groups they can be different or the same. Examples of amino groups include, —NH₂, methylamino (—NH—CH₃); ethylamino (—NHCH₂CH₃), hydroxyethylamino (—NH—CH₂CH₂OH), dimethylamino, methylethylamino, diethylamino, and the like.

h) Mono-Substituted Amino

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

i) Di-Substituted Amino

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

j) Azide

As used herein, the term “azide”, “azido” and their variants refer to any moiety or compound comprising the monovalent group —N₃ or the monovalent ion —N₃.

k) Haloalkyl

The term “haloalkyl” as used herein an alkyl residue as defined above, substituted with one or more halogens, preferably fluorine, such as a trifluoromethyl, pentafluoroethyl and the like.

l) Haloalkoxy

The term “haloalkoxy” as used herein a haloalkyl residue as defined above that is directly attached to an oxygen to form trifluoromethoxy, pentafluoroethoxy and the like.

m) Acyl

The term “acyl” as used herein is a R—C(O)— residue having an R group containing 1 to 8 carbons. The term “acyl” encompass acyl halide, R—(O)-halogen. Examples include but are not limited to formyl, acetyl, propionyl, butanoyl, iso-butanoyl, pentanoyl, hexanoyl, heptanoyl, benzoyl and the like, and natural or un-natural amino acids.

n) Acyloxy

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

o) Aryl

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. An aryl moiety with 1, 2, or 3 alkyl substituent groups can be referred to as “arylalkyl.” It will be understood by those skilled in the art that the moieties substituted on the “aryl” can themselves be substituted, as described above, if appropriate.

p) Heteroaryl

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

q) Heterocyclyl

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

r) Halogen or Halo

The term “halo” or “halogen” refers to a fluoro, chloro, bromo or iodo group.

s) Moiety

A “moiety” is part of a molecule (or compound, or analog, etc.). A “functional group” is a specific group of atoms in a molecule. A moiety can be a functional group or can include one or functional groups.

t) Ester

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

u) Carbonate Group

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

v) Keto Group

The term “keto group” as used herein is represented by the formula —C(O)R, where R is an alkyl, alkenyl, alkynyl, aryl, aralkyl, cycloalkyl, halogenated alkyl, or heterocycloalkyl group described above.

w) Aldehyde

The term “aldehyde” as used herein is represented by the formula —C(O)H or —R—C(O)H, wherein R can be as defined above alkyl, alkenyl, alkoxy, aryl, heteroaryl, cycloalkyl, cycloalkenyl, heterocycloalkyl, or heterocycloalkenyl group described above.

x) Carboxylic Acid

The term “carboxylic acid” as used herein is represented by the formula —C(O)OH.

y) Carbonyl Group

The term “carbonyl group” as used herein is represented by the formula C═O.

z) Ether

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

aa) Urethane

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

bb) Silyl Group

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

cc) Sulfo-Oxo Group

The term “sulfo-oxo group” as used herein is represented by the formulas —S(O)₂R, —OS(O)₂R, or, —OS(O)₂OR, where R can be hydrogen or as defined above an alkyl, alkenyl, alkynyl, aryl, aralkyl, cycloalkyl, halogenated alkyl, or heterocycloalkyl group described above.

24. Clathrate

A compound for use in the invention may form a complex such as a “clathrate”, a drug-host inclusion complex, wherein, in contrast to solvates, the drug and host are present in stoichiometric or non-stoichiometric amounts. A compound used herein can also contain two or more organic and/or inorganic components which can be in stoichiometric or non-stoichiometric amounts. The resulting complexes can be ionised, partially ionised, or non-ionised. For a review of such complexes, see J. Pharm. ScL, 64 (8), 1269-1288, by Haleblian (August 1975).

25. Detect

Detect or like terms refer to an ability of the apparatus and methods of the disclosure to discover or sense a molecule- or a marker-induced cellular response and to distinguish the sensed responses for distinct molecules.

26. Direct Action (of a Drug Candidate Molecule)

A “direct action” or like terms is a result (of a drug candidate molecule”) acting independently on a cell.

27. DMR Signal

A “DMR signal” or like terms refers to the signal of cells measured with an optical biosensor that is produced by the response of a cell upon stimulation.

28. DMR Response

A “DMR response” or like terms is a biosensor response using an optical biosensor. The DMR refers to dynamic mass redistribution or dynamic cellular matter redistribution. A P-DMR is a positive DMR response, a N-DMR is a negative DMR response, and a RP-DMR is a recovery P-DMR response.

29. Drug Candidate Molecule

A drug candidate molecule or like terms is a test molecule which is being tested for its ability to function as a drug or a pharmacophore. This molecule may be considered as a lead molecule.

30. Efficacy

Efficacy or like terms is the capacity to produce a desired size of an effect under ideal or optimal conditions. It is these conditions that distinguish efficacy from the related concept of effectiveness, which relates to change under real-life conditions. Efficacy is the relationship between receptor occupancy and the ability to initiate a response at the molecular, cellular, tissue or system level.

31. hERG Modulator

A hERG modulator is a molecule that can modulate the activity of hERG ion channel directly or indirectly. A hERG modulator that modulates the activity of hERG channel directly is a molecule that binds to hERG channels, thus causing the alteration in hERG activity, such as hERG current, ion flux via hERG, and/or cell signaling via hERG. A hERG modulator that modulates the activity of hERG channel indirectly is a molecule that binds to a hERG-associated signaling complex in cells, thus causing the alteration in hERG activity, such as hERG current, ion flux via hERG, and/or cell signaling via hERG channel or hERG-associated signaling complex. The alteration in hERG activity is referenced to the basal activity of hERG channel or hERG-associated signaling complex in cells in the absence of a modulator.

32. hERG Activator

A hERG activator is a molecule that increases the current via hERG channel at appropriate applied voltages, and/or increases the ion flux via hERG channel in the presence of appropriate KCl concentrations, and/or triggers cell signaling via hERG channel or hERG-associated signaling complex in cells. Examples are mallotoxin, flufenamic acid, and niflumic acid.

33. hERG Pathway Activator

A hERG pathway activator is a molecule that triggers cell signaling via hERG channel or hERG-associated signaling complex in cells. A hERG pathway activator may or may not cause any alteration in hERG current, and/or ion flux via hERG channel. Alteration can either increase or decrease. Examples are diflunisal, AG126, and tyrphostin 51.

34. hERG Ion Channel Activator

A hERG ion channel activator is a molecule that directly binds to and activates hERG channel, thus leading to increase in hERG current, and/or increase in hERG ion flux, and/or cell signaling via hERG channel. Examples are mallotoxin, flufenamic acid, and niflumic acid. A hERG ion channel activator may or may not trigger cell signaling.

35. Label-Free Biosensor hERG Activator

A label-free biosensor hERG activator or like terms is a molecule that is a hERG activator and is capable of triggering a detectable biosensor signal in a hERG expressing cell using a label-free biosensor cellular assay. The biosensor hERG activator can be a hERG activator, a hERG pathway activator, or a hERG ion channel activator. Examples are mallotoxin, RPR260243, NS1643, NS3623, PD-118057, PD-307243, A-935142, flufenamic acid, niflumic acid, or diflunisal.

36. hERG Inhibitor

A hERG inhibitor is a molecule that binds to hERG channel, or hERG-associated signaling complex, thus inhibiting hERG current and/or hERG ion flux.

37. hERG Pathway Inhibitor

A hERG inhibitor is a molecule that binds to hERG-associated signaling complex, thus inhibiting hERG current, and/or hERG ion flux. Example includes tyrphostin 51.

38. hERG Ion Channel Inhibitor

A hERG ion channel inhibitor is a molecule that binds to hERG channel directly and thus inhibits hERG current, and/or hERG ion flux. Example includes dofetilide.

39. Higher and Inhibit and Like Words

The terms higher, increases, elevates, or elevation or like terms or variants of these terms, refer to increases above basal levels, e.g., as compared a control. The terms low, lower, reduces, decreases or reduction or like terms or variation of these terms, refer to decreases below basal levels, e.g., as compared to a control. For example, basal levels are normal in vivo levels prior to, or in the absence of, or addition of a molecule such as an agonist or antagonist to a cell. Inhibit or forms of inhibit or like terms refers to reducing or suppressing.

40. In the Presence of the Molecule

“in the presence of the molecule” or like terms refers to the contact or exposure of the cultured cell with the molecule. The contact or exposure can be taken place before, or at the time, the stimulus is brought to contact with the cell.

41. Index

An index or like terms is a collection of data. For example, an index can be a list, table, file, or catalog that contains one or more modulation profiles. It is understood that an index can be produced from any combination of data. For example, a DMR profile can have a P-DMR, a N-DMR, and a RP-DMR. An index can be produced using the completed date of the profile, the P-DMR data, the N-DMR data, the RP-DMR data, or any point within these, or in combination of these or other data. The index is the collection of any such information. Typically, when comparing indexes, the indexes are of like data, i.e. P-DMR to P-DMR data.

a) Biosensor Index

A “biosensor index” or like terms is an index made up of a collection of biosensor data. A biosensor index can be a collection of biosensor profiles, such as primary profiles, or secondary profiles. The index can be comprised of any type of data. For example, an index of profiles could be comprised of just an N-DMR data point, it could be a P-DMR data point, or both or it could be an impedence data point. It could be all of the data points associated with the profile curve.

b) DMR Index

A “DMR index” or like terms is a biosensor index made up of a collection of DMR data.

42. Known Molecule

A known molecule or like terms is a molecule with known pharmacological/biological/physiological/pathophysiological activity whose precise mode of action(s) may be known or unknown.

43. Known Modulator

A known modulator or like terms is a modulator where at least one of the targets is known with a known affinity. For example, a known modulator could be a PI3K inhibitor, a PKA inhibitor, a GPCR antagonist, a GPCR agonist, a RTK inhibitor, an epidermal growth factor receptor neutralizing antibody, or a phosphodiesterase inhibition, a PKC inhibitor or activator, etc.

44. Known Modulator Biosensor Index

A “known modulator biosensor index” or like terms is a modulator biosensor index produced by data collected for a known modulator. For example, a known modulator biosensor index can be made up of a profile of the known modulator acting on the panel of cells, and the modulation profile of the known modulator against the panels of markers, each panel of markers for a cell in the panel of cells.

45. Known Modulator DMR Index

A “known modulator DMR index” or like terms is a modulator DMR index produced by data collected for a known modulator. For example, a known modulator DMR index can be made up of a profile of the known modulator acting on the panel of cells, and the modulation profile of the known modulator against the panels of markers, each panel of markers for a cell in the panel of cells.

46. Ligand

A ligand or like terms is a substance or a composition or a molecule that is able to bind to and form a complex with a biomolecule to serve a biological purpose. Actual irreversible covalent binding between a ligand and its target molecule is rare in biological systems. Ligand binding to receptors alters the chemical conformation, i.e., the three dimensional shape of the receptor protein. The conformational state of a receptor protein determines the functional state of the receptor. The tendency or strength of binding is called affinity. Ligands include substrates, blockers, inhibitors, activators, and neurotransmitters. Radioligands are radioisotope labeled ligands, while fluorescent ligands are fluorescently tagged ligands; both can be considered as ligands are often used as tracers for receptor biology and biochemistry studies. Ligand and modulator are used interchangeably.

47. Library

A library or like terms is a collection. The library can be a collection of anything disclosed herein. For example, it can be a collection, of indexes, an index library; it can be a collection of profiles, a profile library; or it can be a collection of DMR indexes, a DMR index library; Also, it can be a collection of molecule, a molecule library; it can be a collection of cells, a cell library; it can be a collection of markers, a marker library; A library can be for example, random or non-random, determined or undetermined. For example, disclosed are libraries of DMR indexes or biosensor indexes of known modulators.

48. Marker

A marker or like terms is a ligand which produces a signal in a biosensor cellular assay. The signal is, must also be, characteristic of at least one specific cell signaling pathway(s) and/or at least one specific cellular process(es) mediated through at least one specific target(s). The signal can be positive, or negative, or any combinations (e.g., oscillation). A hERG channel activator, such as mallotoxin, can be a marker for HEK-hERG cells, or HT29 cells, wherein hERG channels are stably expressed, or endogenously expressed in respective cells.

49. Marker Panel

A “marker panel” or like terms is a panel which comprises at least two markers. The markers can be for different pathways, the same pathway, different targets, or even the same targets. For example, mallotoxin can be used as a single marker for both HEK-hERG and HT29 cells. Thus for hERG channel modulator identification and classification, mallotoxin acts as an effective marker panel.

50. Marker Biosensor Index

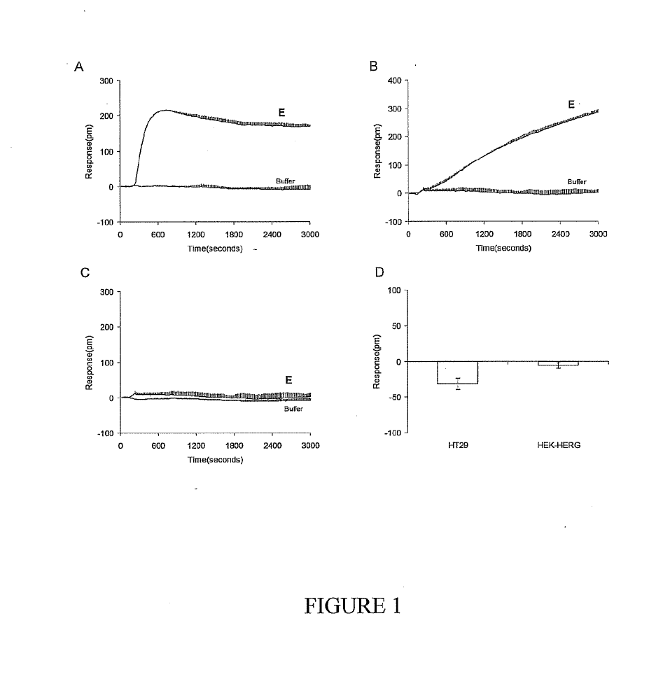

A “marker biosensor index” or like terms is a biosensor index produced by data collected for a marker. For example, a marker biosensor index can be made up of a profile of the marker acting on the panel of cells, and the modulation profile of the marker against the panels of markers, each panel of markers for a cell in the panel of cells. For hERG channel modulator identification and classification, the marker biosensor index includes the primary profiles of a molecule across three different cells (e.g., HEK293, HEK-hERG, and HT29 cells), and the modulation index of the molecule against the mallotoxin DMR signals in both HEK-hERG and HT29 cells, as exampled in FIGS. 1, 2, 5, 6, and 7.

51. Marker DMR Index

A “marker biosensor index” or like terms is a biosensor DMR index produced by data collected for a marker. For example, a marker DMR index can be made up of a profile of the marker acting on the panel of cells, and the modulation profile of the marker against the panels of markers, each panel of markers for a cell in the panel of cells.

52. Material

Material is the tangible part of something (chemical, biochemical, biological, or mixed) that goes into the makeup of a physical object.

53. Mimic

As used herein, “mimic” or like terms refers to performing one or more of the functions of a reference object. For example, a molecule mimic performs one or more of the functions of a molecule.

54. Modulate

To modulate, or forms thereof, means either increasing, decreasing, or maintaining a cellular activity mediated through a cellular target. It is understood that wherever one of these words is used it is also disclosed that it could be 1%, 5%, 10%, 20%, 50%, 100%, 500%, or 1000% increased from a control, or it could be 1%, 5%, 10%, 20%, 50%, or 100% decreased from a control.

55. Modulator

A modulator or like terms is a ligand that controls the activity of a cellular target. It is a signal modulating molecule binding to a cellular target, such as a target protein.

56. Modulation Comparison

A “modulation comparison” or like terms is a result of normalizing a primary profile and a secondary profile.

57. Modulator Biosensor Index

A “modulator biosensor index” or like terms is a biosensor index produced by data collected for a modulator. For example, a modulator biosensor index can be made up of a profile of the modulator acting on the panel of cells, and the modulation profile of the modulator against the panels of markers, each panel of markers for a cell in the panel of cells. As exampled in FIGS. 1, 2, 5, 6, and 7, a hERG modulator biosensor index includes the primary DMR signals in three types of cells (e.g., HT29, HEK-hERG, and HEK293), and the modulation DMR index of the modulator against the mallotoxin DMR signals in both HT29 and HEK-hERG cells.

58. Modulator DMR Index

A “modulator DMR index” or like terms is a DMR index produced by data collected for a modulator. For example, a modulator DMR index can be made up of a profile of the modulator acting on the panel of cells, and the modulation profile of the modulator against the panels of markers, each panel of markers for a cell in the panel of cells. As exampled in FIGS. 1d, 2d, 5d, 6d and 7d, a hERG modulator DMR index is the percentage in modulation of the mallotoxin DMR signals in both HT29 and HEK-hERG cells by the modulator.

59. Modulate the Biosensor Signal of a Marker

Modulate the biosensor signal or like terms is to cause changes of the biosensor signal or profile of a cell in response to stimulation with a marker.

60. Modulate the DMR Signal

Modulate the DMR signal or like terms is to cause changes of the DMR signal or profile of a cell in response to stimulation with a marker.

61. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

62. Molecule Mixture

A molecule mixture or like terms is a mixture containing at least two molecules. The two molecules can be, but not limited to, structurally different (i.e., enantiomers), or compositionally different (e.g., protein isoforms, glycoform, or an antibody with different poly(ethylene glycol) (PEG) modifications), or structurally and compositionally different (e.g., unpurified natural extracts, or unpurified synthetic compounds).

63. Molecule Biosensor Index

A “molecule biosensor index” or like terms is a biosensor index produced by data collected for a molecule. For example, a molecule biosensor index can be made up of a profile of the molecule acting on the panel of cells, and the modulation profile of the molecule against the panels of markers, each panel of markers for a cell in the panel of cells.

64. Molecule DMR Index

A “molecule DMR index” or like terms is a DMR index produced by data collected for a molecule. For example, a molecule biosensor index can be made up of a profile of the molecule acting on the panel of cells, and the modulation profile of the molecule against the panels of markers, each panel of markers for a cell in the panel of cells.

65. Molecule Index

A “molecule index” or like terms is an index related to the molecule.

66. Molecule-Treated Cell

A molecule-treated cell or like terms is a cell that has been exposed to a molecule.

67. Molecule Modulation Index

A “molecule modulation index” or like terms is an index to display the ability of the molecule to modulate the biosensor output signals of the panels of markers acting on the panel of cells. The modulation index is generated by normalizing a specific biosensor output signal parameter of a response of a cell upon stimulation with a marker in the presence of a molecule against that in the absence of any molecule.

68. Molecule Pharmacology

Molecule pharmacology or the like terms refers to the systems cell biology or systems cell pharmacology or mode(s) of action of a molecule acting on a cell. The molecule pharmacology is often characterized by, but not limited, toxicity, ability to influence specific cellular process(es) (e.g., proliferation, differentiation, reactive oxygen species signaling), or ability to modulate a specific cellular target (e.g, hERG channel, hERG-associated, PI3K, PKA, PKC, PKG, JAK2, MAPK, MEK2, or actin).

69. Normalizing

Normalizing or like terms means, adjusting data, or a profile, or a response, for example, to remove at least one common variable. For example, if two responses are generated, one for a marker acting a cell and one for a marker and molecule acting on the cell, normalizing would refer to the action of comparing the marker-induced response in the absence of the molecule and the response in the presence of the molecule, and removing the response due to the marker only, such that the normalized response would represent the response due to the modulation of the molecule against the marker. A modulation comparison is produced by normalizing a primary profile of the marker and a secondary profile of the marker in the presence of a molecule (modulation profile).

70. Optional

“Optional” or “optionally” or like terms means that the subsequently described event or circumstance can or cannot occur, and that the description includes instances where the event or circumstance occurs and instances where it does not. For example, the phrase “optionally the composition can comprise a combination” means that the composition may comprise a combination of different molecules or may not include a combination such that the description includes both the combination and the absence of the combination (i.e., individual members of the combination).

71. Or

The word “or” or like terms as used herein means any one member of a particular list and also includes any combination of members of that list.

72. Profile

A profile or like terms refers to the data which is collected for a composition, such as a cell. A profile can be collected from a label free biosensor as described herein.

a) Primary Profile

A “primary profile” or like terms refers to a biosensor response or biosensor output signal or profile which is produced when a molecule contacts a cell. Typically, the primary profile is obtained after normalization of initial cellular response to the net-zero biosensor signal (i.e., baseline)

b) Secondary Profile

A “secondary profile” or like terms is a biosensor response or biosensor output signal of cells in response to a marker in the presence of a molecule. A secondary profile can be used as an indicator of the ability of the molecule to modulate the marker-induced cellular response or biosensor response.

c) Modulation Profile

A “modulation profile” or like terms is the comparison between a secondary profile of the marker in the presence of a molecule and the primary profile of the marker in the absence of any molecule. The comparison can be by, for example, subtracting the primary profile from secondary profile or subtracting the secondary profile from the primary profile or normalizing the secondary profile against the primary profile.

73. Panel

A panel or like terms is a predetermined set of specimens (e.g., markers, or cells, or pathways). A panel can be produced from picking specimens from a library.

74. Positive Control

A “positive control” or like terms is a control that shows that the conditions for data collection can lead to data collection.

75. Potentiate

Potentiate, potentiated or like terms refers to an increase of a specific parameter of a biosensor response of a marker in a cell caused by a molecule. By comparing the primary profile of a marker with the secondary profile of the same marker in the same cell in the presence of a molecule, one can calculate the modulation of the marker-induced biosensor response of the cells by the molecule. A positive modulation means the molecule to cause increase in the biosensor signal induced by the marker.

76. Potency

Potency or like terms is a measure of molecule activity expressed in terms of the amount required to produce an effect of given intensity. For example, a highly potent drug evokes a larger response at low concentrations. The potency is proportional to affinity and efficacy. Affinity is the ability of the drug molecule to bind to a receptor.

77. Prodrug

“Prodrug” or the like terms refers to compounds that when metabolized in vivo, undergo conversion to compounds having the desired pharmacological activity. Prodrugs may be prepared by replacing appropriate functionalities present in pharmacologically active compounds with “pro-moieties” as described, for example, in H. Bundgaar, Design of Prodrugs (1985). Examples of prodrugs include ester, ether or amide derivatives of the compounds herein, and their pharmaceutically acceptable salts. For further discussions of prodrugs, see e.g., T. Higuchi and V. Stella “Pro-drugs as Novel Delivery Systems,” ACS Symposium Series 14 (1975) and E. B. Roche ed., Bioreversible Carriers in Drug Design (1987).

78. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

79. Receptor

A receptor or like terms is a protein molecule embedded in either the plasma membrane or cytoplasm of a cell, to which a mobile signaling (or “signal”) molecule may attach. A molecule which binds to a receptor is called a “ligand,” and may be a peptide (such as a neurotransmitter), a hormone, a pharmaceutical drug, or a toxin, and when such binding occurs, the receptor goes into a conformational change which ordinarily initiates a cellular response. However, some ligands merely block receptors without inducing any response (e.g. antagonists). Ligand-induced changes in receptors result in physiological changes which constitute the biological activity of the ligands.

80. “Robust Biosensor Signal”

A “robust biosensor signal” is a biosensor signal whose amplitude(s) is significantly (such as 3×, 10×, 20×, 100×, or 1000×) above either the noise level, or the negative control response. The negative control response is often the biosensor response of cells after addition of the assay buffer solution (i.e., the vehicle). The noise level is the biosensor signal of cells without further addition of any solution. It is worthy of noting that the cells are always covered with a solution before addition of any solution.

81. “Robust DMR Signal”

A “robust DMR signal” or like terms is a DMR form of a “robust biosensor signal.”

82. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

83. Response

A response or like terms is any reaction to any stimulation.

84. Sample

By sample or like terms is meant an animal, a plant, a fungus, etc.; a natural product, a natural product extract, etc.; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

85. Salt(s) and Pharmaceutically Acceptable Salt(s)

The compounds of this invention may be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil. In some instances, a salt of a compound also may be used as an aid in the isolation, purification, and/or resolution of the compound.

Where a salt is intended to be administered to a patient (as opposed to, for example, being used in an in vitro context), the salt preferably is pharmaceutically acceptable. The term “pharmaceutically acceptable salt” refers to a salt prepared by combining a compound of formula I or II with an acid whose anion, or a base whose cation, is generally considered suitable for human consumption. Pharmaceutically acceptable salts are particularly useful as products of the methods of the present invention because of their greater aqueous solubility relative to the parent compound. For use in medicine, the salts of the compounds of this invention are non-toxic “pharmaceutically acceptable salts.” Salts encompassed within the term “pharmaceutically acceptable salts” refer to non-toxic salts of the compounds of this invention which are generally prepared by reacting the free base with a suitable organic or inorganic acid.

Suitable pharmaceutically acceptable acid addition salts of the compounds of the present invention when possible include those derived from inorganic acids, such as hydrochloric, hydrobromic, hydrofluoric, boric, fluoroboric, phosphoric, metaphosphoric, nitric, carbonic, sulfonic, and sulfuric acids, and organic acids such as acetic, benzenesulfonic, benzoic, citric, ethanesulfonic, fumaric, gluconic, glycolic, isothionic, lactic, lactobionic, maleic, malic, methanesulfonic, trifluoromethanesulfonic, succinic, toluenesulfonic, tartaric, and trifluoroacetic acids. Suitable organic acids generally include, for example, aliphatic, cycloaliphatic, aromatic, araliphatic, heterocyclic, carboxylic, and sulfonic classes of organic acids.

Specific examples of suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), methanesulfonate, ethanesulfonate, benzenesulfonate, pantothenate, toluenesulfonate, 2-hydroxyethanesulfonate, sufanilate, cyclohexylaminosulfonate, algenic acid, β-hydroxybutyric acid, galactarate, galacturonate, adipate, alginate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate. Furthermore, where the compounds of the invention carry an acidic moiety, suitable pharmaceutically acceptable salts thereof may include alkali metal salts, i.e., sodium or potassium salts; alkaline earth metal salts, e.g., calcium or magnesium salts; and salts formed with suitable organic ligands, e.g., quaternary ammonium salts. In another embodiment, base salts are formed from bases which form non-toxic salts, including aluminum, arginine, benzathine, choline, diethylamine, diolamine, glycine, lysine, meglumine, olamine, tromethamine and zinc salts.

Organic salts may be made from secondary, tertiary or quaternary amine salts, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups may be quaternized with agents such as lower alkyl (CrC₆) halides (e.g., methyl, ethyl, propyl, and butyl chlorides, bromides, and iodides), dialkyl sulfates (i.e., dimethyl, diethyl, dibuytl, and diamyl sulfates), long chain halides (i.e., decyl, lauryl, myristyl, and stearyl chlorides, bromides, and iodides), arylalkyl halides (i.e., benzyl and phenethyl bromides), and others.

In one embodiment, hemisalts of acids and bases may also be formed, for example, hemisulphate and hemicalcium salts.

The compounds of the invention and their salts may exist in both unsolvated and solvated forms.

86. Signaling Pathway(s)

A “defined pathway” or like terms is a path of a cell from receiving a signal (e.g., an exogenous ligand) to a cellular response (e.g., increased expression of a cellular target). In some cases, receptor activation caused by ligand binding to a receptor is directly coupled to the cell's response to the ligand. For example, the neurotransmitter GABA can activate a cell surface receptor that is part of an ion channel. GABA binding to a GABA A receptor on a neuron opens a chloride-selective ion channel that is part of the receptor. GABA A receptor activation allows negatively charged chloride ions to move into the neuron which inhibits the ability of the neuron to produce action potentials. However, for many cell surface receptors, ligand-receptor interactions are not directly linked to the cell's response. The activated receptor must first interact with other proteins inside the cell before the ultimate physiological effect of the ligand on the cell's behavior is produced. Often, the behavior of a chain of several interacting cell proteins is altered following receptor activation. The entire set of cell changes induced by receptor activation is called a signal transduction mechanism or pathway. The signaling pathway can be either relatively simple or quite complicated.

87. Similarity of Indexes

“Similarity of indexes” or like terms is a term to express the similarity between two indexes, or among at least three indices, one for a molecule, based on the patterns of indices, and/or a matrix of scores. The matrix of scores are strongly related to their counterparts, such as the signatures of the primary profiles of different molecules in corresponding cells, and the nature and percentages of the modulation profiles of different molecules against each marker. For example, higher scores are given to more-similar characters, and lower or negative scores for dissimilar characters. Because there are only three types of modulation, positive, negative and neutral, found in the molecule modulation index, the similarity matrices are relatively simple. For example, a simple matrix will assign identical modulation (e.g., a positive modulation) a score of +1 and non-identical modulation a score of −1.

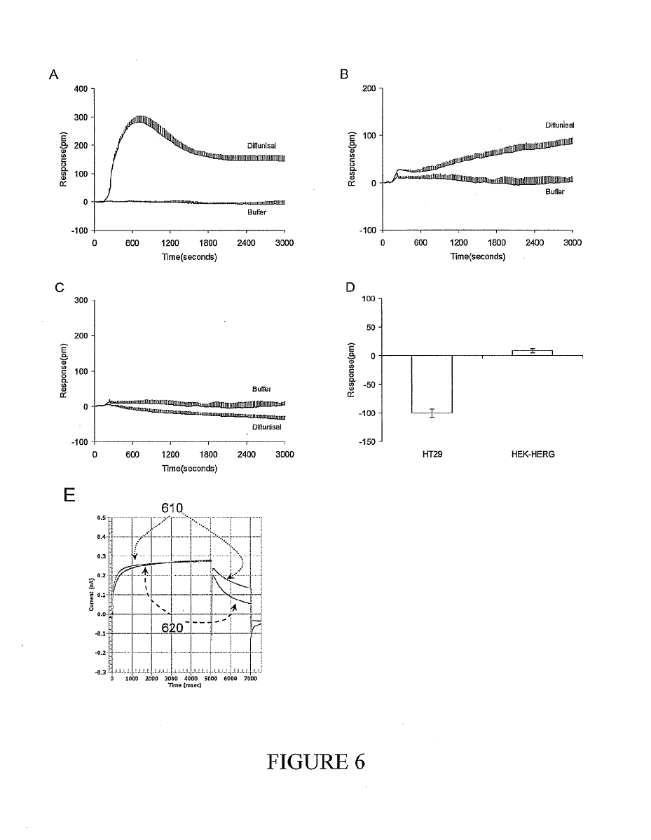

Alternatively, different scores can be given for a type of modulation but with different scales. For example, a positive modulation of 10%, 20%, 30%, 40%, 50%, 60%, 100%, 200%, etc, can be given a score of +1, +2, +3, +4, +5, +6, +10, +20, correspondingly. Conversely, for negative modulation, similar but in opposite score can be given. Following this approach, the modulation index of flufenamic acid against mallotoxin in the two cells, as shown in FIG. 5d, illustrates that flufenamic acid modulates differently the biosensor response induced by mallotoxin in the two cells: HT29 (−90%), and HEK-hERG (˜+12%). Thus, the score of flufenamic acid modulation index in coordination can be assigned as (−9, 1). Similarly, for diflunisal its score in coordination is (−9, 1) (FIG. 6d). By comparing the scores between flufenamic acid and diflunisal, one can conclude that both molecules exhibits similar mode(s) of action acting on hERG channels

88. Solvate

The compounds herein, and the pharmaceutically acceptable salts thereof; may exist in a continuum of solid states ranging from fully amorphous to fully crystalline. They may also exist in unsolvated and solvated forms. The term “solvate” describes a molecular complex comprising the compound and one or more pharmaceutically acceptable solvent molecules (e.g., EtOH). The term “hydrate” is a solvate in which the solvent is water. Pharmaceutically acceptable solvates include those in which the solvent may be isotopically substituted (e.g., D₂O, d₆-acetone, d₆-DMSO).

A currently accepted classification system for solvates and hydrates of organic compounds is one that distinguishes between isolated site, channel, and metal-ion coordinated solvates and hydrates. See, e.g., K. R. Morris (H. G. Brittain ed.) Polymorphism in Pharmaceutical Solids (1995). Isolated site solvates and hydrates are ones in which the solvent (e.g., water) molecules are isolated from direct contact with each other by intervening molecules of the organic compound. In channel solvates, the solvent molecules lie in lattice channels where they are next to other solvent molecules. In metal-ion coordinated solvates, the solvent molecules are bonded to the metal ion.

When the solvent or water is tightly bound, the complex will have a well-defined stoichiometry independent of humidity. When, however, the solvent or water is weakly bound, as in channel solvates and in hygroscopic compounds, the water or solvent content will depend on humidity and drying conditions. In such cases, non-stoichiometry will be the norm.

The compounds herein, and the pharmaceutically acceptable salts thereof, may also exist as multi-component complexes (other than salts and solvates) in which the compound and at least one other component are present in stoichiometric or non-stoichiomethc amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallization, by recrystallization from solvents, or by physically grinding the components together. See, e.g., O. Almarsson and M. J. Zaworotko, Chem. Commun., 17:1889-1896 (2004). For a general review of multi-component complexes, see J. K. Haleblian, J. Pharm. Sci. 64(8):1269-88 (1975).

89. Stable

When used with respect to pharmaceutical compositions, the term “stable” or like terms is generally understood in the art as meaning less than a certain amount, usually 10%, loss of the active ingredient under specified storage conditions for a stated period of time. The time required for a composition to be considered stable is relative to the use of each product and is dictated by the commercial practicalities of producing the product, holding it for quality control and inspection, shipping it to a wholesaler or direct to a customer where it is held again in storage before its eventual use. Including a safety factor of a few months time, the minimum product life for pharmaceuticals is usually one year, and preferably more than 18 months. As used herein, the term “stable” references these market realities and the ability to store and transport the product at readily attainable environmental conditions such as refrigerated conditions, 2° C. to 8° C.

90. Substance

A substance or like terms is any physical object. A material is a substance. Molecules, ligands, markers, cells, proteins, and DNA can be considered substances. A machine or an article would be considered to be made of substances, rather than considered a substance themselves.

91. Subject

As used throughout, by a subject or like terms is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) and mammals, non-human mammals, primates, non-human primates, rodents, birds, reptiles, amphibians, fish, and any other animal. In one aspect, the subject is a mammal such as a primate or a human. The subject can be a non-human.

92. Test Molecule

A test molecule or like terms is a molecule which is used in a method to gain some information about the test molecule. A test molecule can be an unknown or a known molecule.

93. Treating

Treating or treatment or like terms can be used in at least two ways. First, treating or treatment or like terms can refer to administration or action taken towards a subject. Second, treating or treatment or like terms can refer to mixing any two things together, such as any two or more substances together, such as a molecule and a cell. This mixing will bring the at least two substances together such that a contact between them can take place.

When treating or treatment or like terms is used in the context of a subject with a disease, it does not imply a cure or even a reduction of a symptom for example. When the term therapeutic or like terms is used in conjunction with treating or treatment or like terms, it means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease.

94. Trigger

A trigger or like terms refers to the act of setting off or initiating an event, such as a response.

95. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

96. Unknown Molecule

An unknown molecule or like terms is a molecule with unknown biological/pharmacological/physiological/pathophysiological activity.

97. Optimizing

Optimizing refers to a process of making better or checking to see if it something or some process can be made better.

98. Therapeutic Efficacy

Therapeutic efficacy refers to the degree or extent of results from a treatment of a subject.

99. Disease Marker

A disease marker is any reagent, molecule, substance etc, that can be used for identifying, diagnosing, or prognosing is for the hERG channel related disease.

100. hERG Channel Related Disease

A hERG channel related disease is a disease in which the cause of the disease or the treatment of the disease can be altered by modulation of the hERG channel. Exemplary diseases are cancers, such as leukemia, colon cancer, gastric cancer, breast cancer, or lung cancer. Exemplary diseases are genetic mutation caused inherited long QT syndrome (LQTS), drug molecule-caused acquired LQTS, and class III arrhymics.

101. Toxicity Marker

A toxicity marker is any reagent, molecule, substance etc. that can be used for identifying, diagnosing, prognosing a level of toxicity of a substance, in for example, an organism or cell or tissue or organ.

102. Analytical Methods

An analytical method is for example, a method which measures a molecule or substance. For example, gas chromatography, gel permeation chromatography, high resolution gas chromoatography, high resolution mass spectrometry, or mass spectrometry is analytical methods.

103. Toxicity

Toxicity is the degree to which a substance, molecule, is able to damage something, such as a cell, a tissue, an organ, or a whole organism, that has been exposed to the substance or molecule. For example, the liver, or cells in the liver, hepatocytes, can be damaged by certain substances.

### B. VOLTAGE-DEPENDENT ION CHANNELS

Voltage-dependant ion channels are proteins that span cell surface membranes in excitable tissue such as heart and nerves. Ions passing through channels form the basis of the cardiac action potential. Influx of Na⁺ and Ca²⁺ ions, respectively, control the depolarizing upstroke and plateau phases of the action potential. K⁺ ion efflux repolarizes the cell membrane, terminates the action potential, and allows relaxation of the muscle. A rapid component of the repolarizing current flows through the K+ channel encoded by the human ether-à-go-go-related gene (hERG). Impaired repolarization can prolong the duration of the action potential, delay relaxation and promote disturbances of the heartbeat. Action potential prolongation is detected clinically as a lengthening of the QT interval measured on the electrocardiogram (ECG). Drug-induced QT prolongation is a serious complication of drugs due to impaired repolarization, which is associated with an increased risk of lethal ventricular arrhythmias. Drug-induced QT prolongation is almost always associated with block of the hERG K+ channel. A plethora of drugs, such as methanesulfonanilides, dofetilide, MK-499, and E-4031 are known to block K⁺ion channels such as hERG on the heart causing a life threatening ventricular arrhythmia and heart attack in susceptible individuals. Unfortunately, incidence of drug-induced ventricular arrhythmia is often too low to be detected in clinical trials.

A sudden death due to the blocking of hERG channels by noncardiovascular drugs such as terfenadine (antihistamine), astemizole (antihistamine), and cisapride (gastrokinetic) led to their withdrawal from the market. Recently, drugs like Vioxx were also pulled out of the market for concerns relating to dangerous cardiac side effects. Consequently, cardiac safety relating to K⁺ channels has become a major concern of regulatory agencies. In order to prevent costly attrition, it has therefore become a high priority in drug discovery to screen out inhibitory activity on hERG channels in lead compounds as early as possible.

Current methods for testing potential drug molecules for hERG blocking activity have several limitations. Technologies based on cell-based patch clamp electrophysiology or animal tests are technically difficult and do not meet the demand for throughput and precision for preclinical cardiac safety tests. Other assays use radio-labeled, fluorescent, dye-conjugated, or biotinylated markers for detection and quantification of binding. However, many of these markers have reduced activity after labeling. In addition, the use of radio-labeled analogs poses practical limitations such as requirements for complex infrastructure and licenses for operating radioactive compounds. The promiscuous nature of this channel, referred to herein as the hERG K⁺ channel, or hERG, or hERG ion channel, or hERG channel, leads to it binding a diverse set of chemical structures (Cavalli, A et al., J Med Chem 2002, 45(18), 3844-53), coupled with the potential fatal outcome that may emerge from that interaction. These realities have resulted in the recommendation from the International Congress of Harmonization and the U.S. Food and Drug Administration that all new drug candidates undergo testing in a functional patch-clamp assay using the human hERG protein, either in native form or expressed in recombinant form (Bode, G., et al., Fundam Clin Pharmacol 2002, 16(2), 105-18). Although automated, high-throughput patch-clamp methods have been recently developed, such systems require specialized operators, live cells, and a substantial capital investment (Bridgland-Taylor, M. et al., J. Pharmacol. Toxicol. Methods 2006, 54(2), 189-99; Dubin, A. et al., J. Biomol. Screen. 2005, 10, (2), 168-81). Accordingly, there is a need to develop new compositions and methods for characterizing and quantifying the binding of molecules, such as drug candidates, to hERG channels.

The KCNH2 or human-Ether-à-go-go Related Gene (hERG) encodes Kv11.1 α-subunits that combine to form Kv11.1 potassium channels. The hERG gene is translated as a core-glycosylated immature 135 kDa protein (Kv11.1) in the endoplasmic reticulum and is converted to a complexly-glycosylated mature 155 kDa protein in the Golgi apparatus. (Warmke, J. W., et al. Proc. Natl. Acad. Sci. 1994. 91(8), 3438-3442 incorporated by reference) discloses the sequence and structure of the hERG gene and its wild type translation product, Kv11.1. The sequence of hERG protein is disclosed in SEQ ID NO:1 (NP_{—}00229). The sequence of hERG gene is disclosed in SEQ ID NO:2 (NM_{—}00238)

Disclosed are hERG modulators, hERG activator, label-free biosensor hERG activator, hERG pathway activator, hERG ion channel activator, hERG inhibitor, hERG pathway inhibitor, and hERG ion channel inhibitor. A hERG ion channel activator that can override a drug molecule caused alteration in hERG current can be beneficial clinically, due to reduced hERG liability. Such hERG ion channel activator can be used as a drug combination with a drug molecule who is also a hERG blocker. A hERG pathway inhibitor that does not cause any alteration in hERG current could also be beneficial, particularly as an anti-cancer agent. Such hERG pathway blocker has minimal hERG liability, and also has anti-proliferative activity against cancers.

The disclosed methods and compounds are useful as therapeutic agents for modulating hERG ion channels, and for improved prevention and treatment of hERG associated cardiac repolarization disorders. The compounds' structural formula or a pharmaceutically acceptable sale, solvate, clathrate, or prodrug thereof, wherein R⁶, R⁵, R⁴, X and Y are defined herein.

The disclosed compounds relate to 2-dicyanomethyl-3-cyano-2,5-dihydrofuran and derivatives thereof, as described in formula (I) and (II) and pharmaceutically acceptable salts, solvates, clathrates, and prodrugs thereof, wherein R¹, R², R³, R⁶, R⁵, and R⁴ are defined herein.

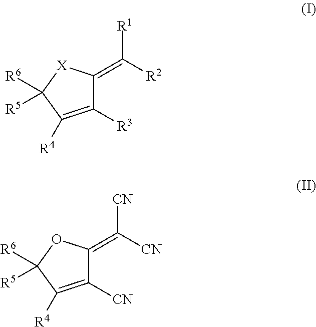

R⁶ and R⁵ can independently be selected from —H, unsubstituted or substituted alkyl, unsubstituted or substituted alkynyl, unsubstituted or substituted alkenyl, unsubstituted or substituted aryl, unsubstituted or substituted alkylaryl, unsubstituted or substituted carbocycle, unsubstituted or substituted heterocycle, unsubstituted or substituted cyclohexyl, and (CH2)n-O—(CH2)n, where n is 1-10.

R4 can independently be selected from alkyl, or double bond connected fully conjugated chromophore with electronic donating-bridge-accepting structure or only donating-accepting or bridge-accepting structures.

Preferred electron donating groups are described in, for example, U.S. Pat. Nos. 6,393,190B1, 5,044,725, 4,795,664, 5,247,042, 5,196,509, 4,810,338, 4,936,645, 4,767,169, 5,326,661, 5,187,234, 5,170,461, 5,133,037, 5,106,211, and 5,006,285, each of which is incorporated herein by reference in its entirety. Preferably, the electron donating group is selected from the group consisting of, but not limited to, phenyl ring(s) substituted in the para position by, for example, amino, alkylamino, dialkylamino, dialkylanilino, 1-piperidino, 1-piperazino, 1-pyrrolidino, acylamino, hydroxyl, thiolo, alkylthio, arylthio, alkoxy, aryloxy, acyloxy, alkyl, vinyl, 1,2,3,4-tetrahydroquinolinyl, and the like.

Preferred bridge group is a cyclic bridge which couples the substituted or unsubstituted 2-dicyanomethyl-3-cyano-2,5-dihydrofuran and the electron donating group. Preferably, the bridge group is at least one bivalent ring. Preferred cyclic bridges comprise one or a plurality of bivalent rings. Preferred bivalent rings which can be employed as cyclic bridges in the present application are described in, for example, U.S. Pat. Nos. 6,393,190B1, 5,044,725, 4,795,664, 5,247,042, 5,196,509, 4,810,338, 4,936,645, 4,767,169, 5,326, 661, 5,187,234, 5,170,461, 5,133,037, 5,106,211, and 5,006,285, each of which is incorporated herein by reference in its entirety. Ring bridge can be aromatic or non-aromatic.

A compound of the invention or a pharmaceutically acceptable salt, solvate, clathrate, or prodrug thereof is particularly useful activating hERG ion channels. In particular, a compound of the invention or a pharmaceutically acceptable salt, solvate, clathrate, or prodrug thereof can override the potential LQTS induced by hERG blockers, thus improve the prevention and treatment of hERG associated cardiac repolarization disorders.

Disclosed are compounds or a pharmaceutically accepted salt, solvate, clthrate, or prodrug thereof useful for characterizing hERG ion channels both in vivo and in vitro, and developing drugs that have reduced liability in cardiovascular safety.

Also disclosed are pharmaceutical compositions comprising an effective amount of a compound or a pharmaceutically accepted salt, solvate, clathrate, or prodrug thereof; and a pharmaceutically acceptable carrier or vehicle. These compositions may further comprise additional agents. These compositions are useful for treating or preventing hERG associated cardiac repolarization disorders.

Also disclosed are methods for treating or preventing hERG associated cardiac repolarization disorders, comprising administering to a subject in need thereof a compound of the invention or a pharmaceutically acceptable salt, solvate, clathrate, or prodrug thereof, or a pharmaceutical composition comprising a compound of the invention or a pharmaceutically acceptable salt, solvate, clathrate, or prodrug thereof. These methods may also comprise administering to the subject an additional agent separately or in a combination composition with the compound of the invention or a pharmaceutically acceptable salt, solvate, clathrate, or prodrug thereof.

Also disclosed are methods for modulating hERG ion channels and the cell functions including cancer cell proliferation, in vivo or in vitro using an effective amount of a compound of the invention, or a pharmaceutically acceptable salt, solvate, clathrate or prodrug thereof, or a pharmaceutical composition comprising an effective amount of a compound of the invention or a pharmaceutically acceptable salt, solvate, clathrate or prodrug thereof.

All of the disclosed methods can be practiced with a compound alone, or in combination with other agents, such as hERG blockers or other drugs that could cause LQTS.

Disclosed herein are compositions and methods for modulating hERG ion channel in a subject, comprising administering one or more compounds chosen from:

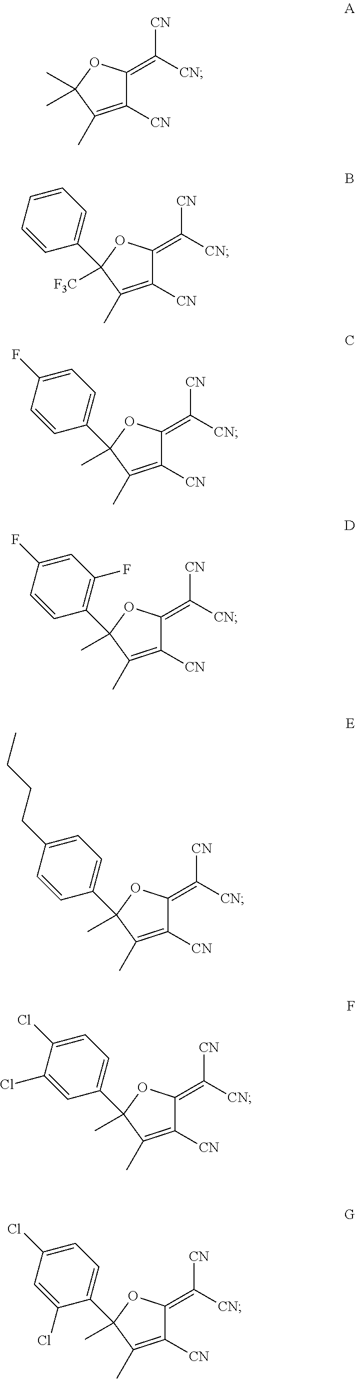

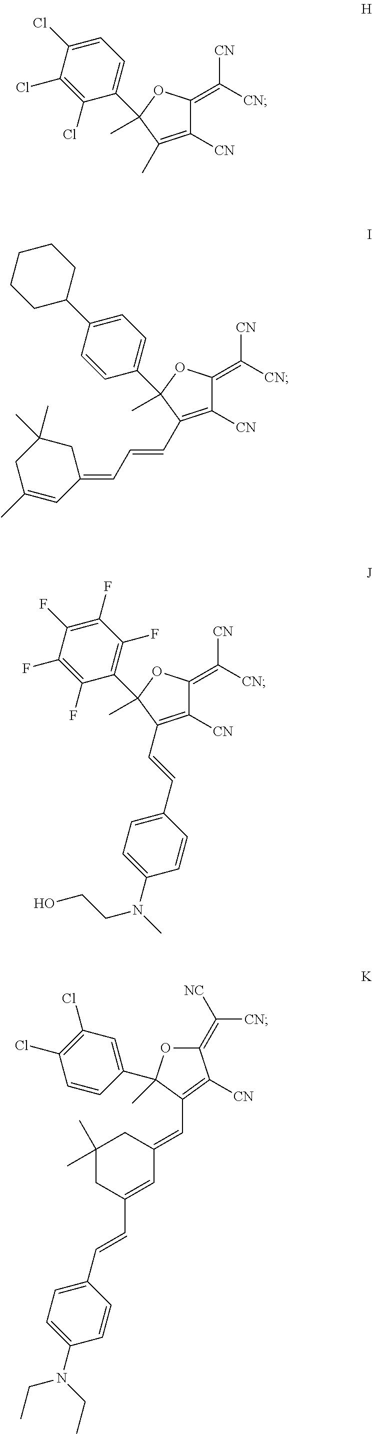

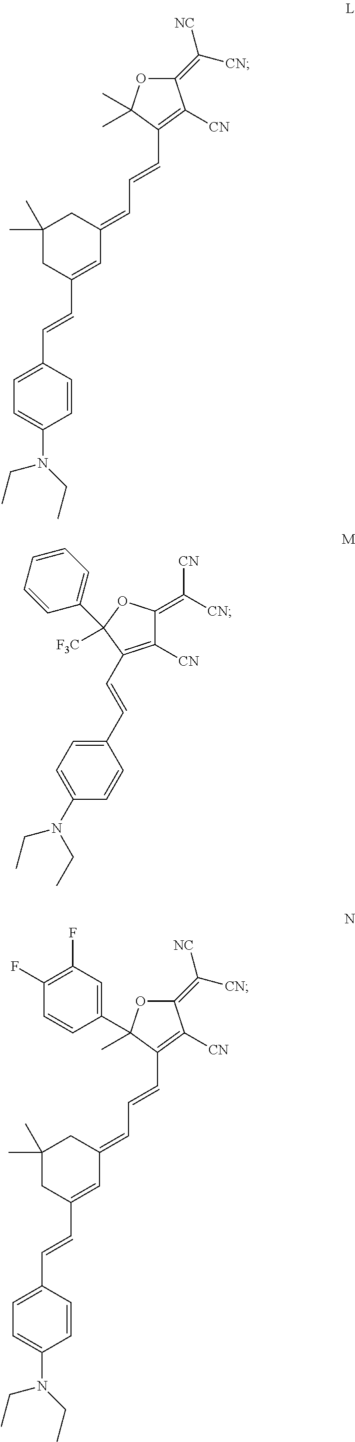

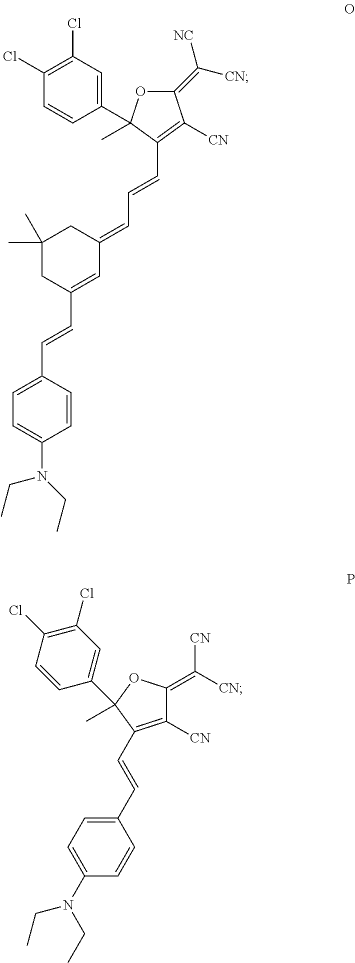

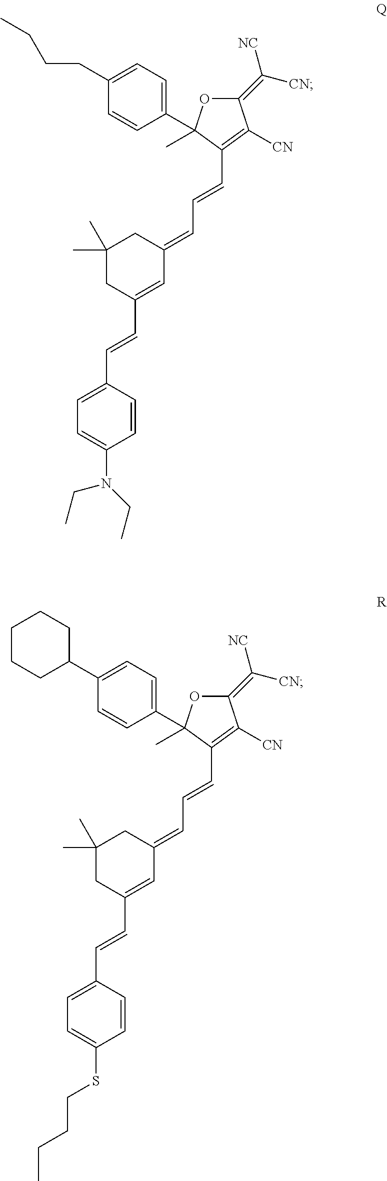

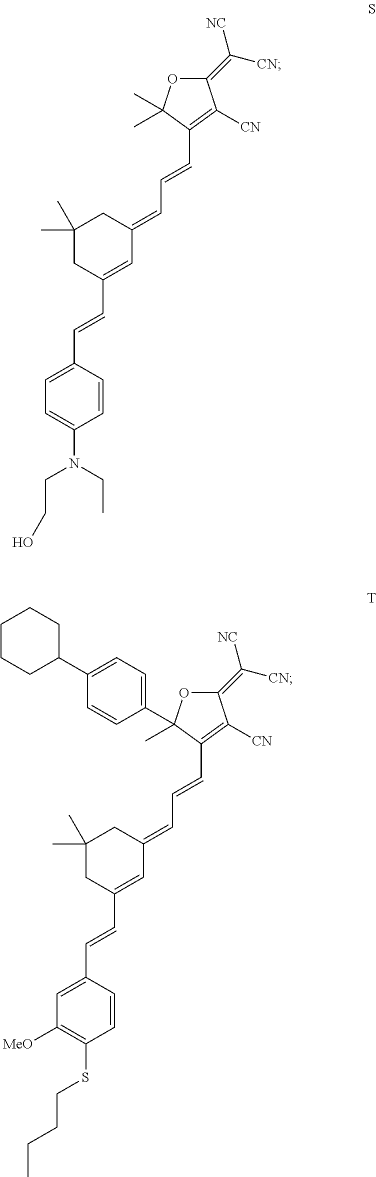

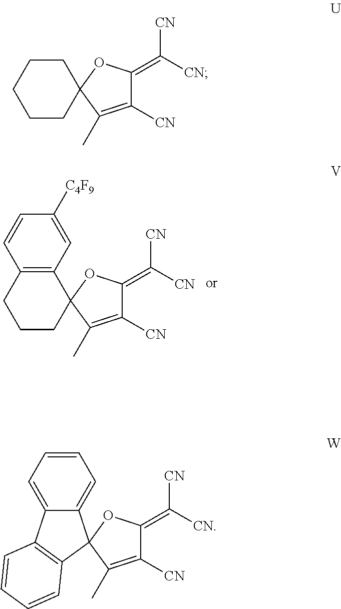

### II. EXAMPLES

### A. Example 1

### Chemical Synthesis and Characterization

### Compound E: 2-dicyanomethylene-3-cyano-4,5-dimethyl-5-[4′-n-butylphenyl]-2,5-dihydrofuran

3-hydroxy-3-[4′-n-butylphenyl]-2-butanone 10.0 g (0.0455 mole), malononitrile 6.0 g (0.091 mole), lithium ethoxide 2.3 ml (2.3 mmole) and THF 50 ml were mixed and boiled at reflux overnight. Pure E was obtained from recrystallization in ethanol to give 9.95 g: yield 69.1%. mp: 129.3-130.5° C. ¹H NMR: δ 7.259 (d, 2H), 7.112 (d, 2H), 2.638 (t, 2H, CH₂), 2.225 (s, 3H, Me), 1.997 (s, 3H, Me), 1.60-1.28 (m, 4H, CH₂), 0.931 (t, 3H, CH₃). ¹³C NMR: 182.476, 175.753, 145.841, 131.069, 129.692 (2C), 125.075 (2C), 110.914, 110.406, 109.088, 104.728, 101.724, 58.830, 35.236, 33.277, 22.335 (2C),14.558, 13.873. HPLC: 100%.

### Compound D: 2-dicyanomethylene-3-cyano-4,5-dimethyl-5-[2′,4′-difluorophenyl]-2,5-dihydrofuran

3-hydroxy-3-[2′,4′-difluorophenyl]-2-butanone 8 g (0.04 mole), malononitrile 5.3 g (0.08 mole) and lithium ethoxide 2 ml (2 mmole) were refluxed in 50 ml of THF overnight. After workup, pure D was obtained by crystallization from ethanol. It afforded 4.5 grams: yield 37.9%. Mp: 219.0-221.4. ¹H NMR: δ 7.441 (d, 1H), 7.066 (dd, 1H), 6.945 (d, 1H), 2.274 (s, 3H, Me), 2.041 (s, 3H, Me). ¹⁹F NMR: −104.81 (d, 1F), 107.294 (d, 1F). HPLC: 100%.

### Compound Q: 3-Cyano-2-(dicyanomethylidene)-4-{trans,trans,trans-[3-(2-(4-(N,N-diethylamino)-phenyl)vinyl)cyclohex-2-enylidene]-1-propenyl}-5-methyl-5-(4-n butylphenyl)-2,5-dihydrofuran

2-[3-[2-[4-(diethylamino)phenyl]ethenyl]-5,5-dimethyl-2-cyclohexen-1-ylidene]-Acetaldehyde, 3.0 g (9.3 mmol), and 2-(dicyanomethylene)-3-cyano-4,5-dimethyl-5-(4-n-butylphenyl)-2,5-dihydrofuran, 3.5 g (11.2 mmol), were reacted in THF/ethanol and purified using column chromatography silica gel (ethyl acetate 20% in hexane). After chromatography, 1.5 g of Q was obtained. Yield: 26.0%. ¹H NMR (solvent CD₂Cl₂): δ 7.31 (m, 7), (m, 2), 6.62 (m, 2H), 6.37 (m, 2H), 6.25 (m, 1H), 3.40 (q, 4H), 2.64 (t, 2H), 2.34 (s, 2H), 2.09 (m, 2H), 2.07 (s, 3H), 1.60-1.36 (m, 4H), 1.17 (t, 6H), 1.00 (s, 3H), 0.92 (t, 3H), 0.85 (s, 3H). ¹³C NMR: 177.16, 174.00, 156.19, 149.74, 149.23, 146.25, 145.94, 134.94, 134.27, 129.94, 129.77, 129.61, 127.86, 126.63, 125.88, 124.35, 116.27, 113.19, 112.71, 112.20, 98.92, 95.24, 55.32, 45.06, 39.84, 35.83, 34.08, 31.73, 29.18, 28.05, 22.91, 14.24, 13.00. Molecular formula: C₄₂H₄₆N₄O. Exact mass+Na: 645.3569 (calculated). 645.3559 (observed). Deviation (ppm): 0.6.

### Compound U: 2-dicyanomethylene-3-cyano-4-methyl-5-spiro-cyclohexyl-2,5-dihydrofuran

1-hydroxy-1-cyclohexyl-ethanone 14.2 g (0.1 mole), malononitrile 13.2 g (0.2 mole), Sodium ethoxide 100 ml (0.1 mole, 1M solution in ethanol) and ethanol 100 ml were mixed and reacted overnight at room temperature. The pure U was obtained from crystallization in ethanol to give 16.1 grams: yield 67.5%. mp: 236.5-237.5° C. ¹H NMR: δ 2.337 (s, 3H, Me), 1.86-1.70 (m, 11H, ring). ¹³C NMR: 182.387, 175.230, 110.980, 110.458, 108.999, 104.883, 101.437, 58.572, 33.123 (2C), 23.918, 21.289 (2C), 14.499. HPLC: 100%.

### Compound B: 2-dicyanomethylene-3-cyano-4-methyl-5-phenyl-5-perfluoromethyl-2,5-dihydrofuran

3-hydroxy-3-phenyl-4,4,4-trifluoro-2-butanone 10.0 g (0.046 mole), malononitrile 6.1 g (0.092 mole), lithium ethoxide 2.5 ml (2.5 mmole) and THF 20 ml were mixed and refluxed overnight. The pure B was obtained through a column chromatography (100% dichloromethane on silica gel, 60-200 mesh) to give 3.75 grams: yield 25.9%. mp: 133-135° C. ¹H NMR: 7.577-7.546 (m, 3H, Ar), 7.444-7.410 (m, 2H, Ar), 2.479 (s, 3H, Me). ¹⁹F NMR: −72.852. ¹³C NMR: 174.224, 172.148, 131.908, 130.191, 127.467, 125.742, 121.682, 109.793, 109.511, 109.098, 108.099, 98.521, 62.938, 15.439. GC/MS: 317 (M+2), 247 (M-CF3). HPLC: 100%.

### Compound V: 2-dicyanomethylene-3-cyano-4-methyl-5-spiro-[7-(1′,1′,2′,2′,3′,3′,4′,4′,4′-perfluorobutyl)tetralin]-2,5-dihydrofuran

1-hydroxy-1-acetyl-7-[1′,1′,2′,2′,3′,3′,4′,4′,4′-nonafluorobutyl] tetralin 6.8 g (0.0167 mole), malononitrile 2.2 g (0.033 mole), lithium ethoxide 1 ml (1 mmole, 1M solution in ethanol) and THF 20 ml were reacted and refluxed overnight. Pure V was obtained by crystallization from ethanol to give 1 g: yield 11.86%. mp: 211.0-212.4° C. ¹H NMR: δ 7.636 (dd, 1H), 7.479 (d, 1H), 6.925 (d, 1H), 3.148-2.879 (m, 2H), 2.252 (s, 3H, Me), 2.166 (m, 4H). ¹³C NMR: 178.962, 173.437, 142.286, 130.305, 128.206, 127.779, 127.473, 124.453, 118.514, 116.378, 114.119 (2C), 109.570, 109.105 (2C), 107.892, 107.830, 106.571, 98.558, 58.921, 32.632, 27.877, 17.537, 13.797. ¹⁹F NMR: −111.843 (2F), −123.282 (2F), −126.127 (3F). HPLC: 100%.

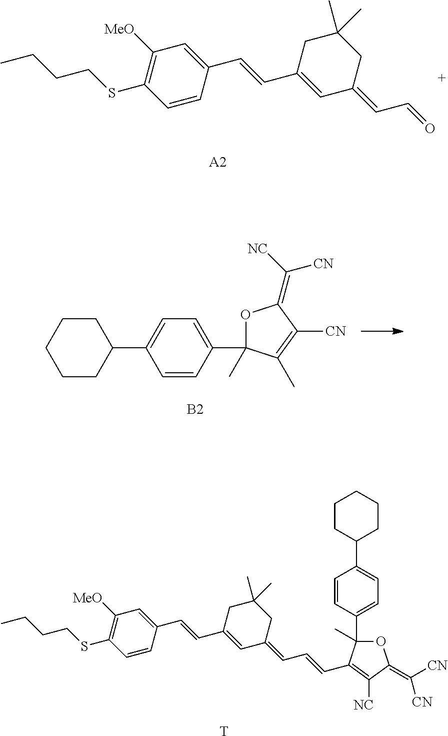

### Compound T

To a mixture of A2 3.0 g (0.008 mole) and B2 2.9 g (0.008 mole), 20 mL of THF and 30 mL of EtOH were added. While this mixture was heated at reflux, 5 drops of anhydrous piperidine were added. This mixture was heated at this temperature for 16 hours. The reaction mixture was then cooled to room temperature and solvents were removed under reduced pressure to yield an oil which was dissolved in methylene chloride. This methylene chloride solution was washed by water and dried over anhydrous MgSO4 and concentrated to form an oily product. The pure T was obtained through a column chromatography (100% methylene chloride on silica to give 3.0 grams: yield 54%. HPLC: 100%.

### Compound W: 2-dicyanomethylene-3-cyano-4-methyl-5-spiro-fluorenylidine-2,5-dihydrofuran

9-hydroxy-9-acetyl-fluorene 5.0 g (0.0223 mole), malononitrile 2.94 g (0.0446 mole), anhydrous potassium carbonate 3.1 g (0.0223 mole), 18-crown-6 ether (catalytic amount) and dry THF 50 ml were mixed and refluxed over night. The pure 041 was collected by crystallization from ethanol to give 3.22 g: yield 45.0%. mp: 302-303° C. ¹H NMR: δ 7.760 (d, 2H), 7.566 (t, 2H), 7.393 (t, 2H), 7.187 (d, 2H), 1.935 (s, 3H). ¹³C NMR: 177.504, 140.459. HPLC: 100%.

### Compound G: 2-dicyanomethylene-3-cyano-4,5-dimethyl-5-[2′,4′-dichlorophenyl]-2,5-dihydrofuran

1-hydroxy-1-methyl-1-[2′,4′-dichlorophenyl]-2-propanone 6.8 g (0.029 mole), malononitrile 3.8 g (0.058 mole), lithium ethoxide 1 ml (1 mmole) and THF 10 ml were mixed and boiled at reflux for 36 hours. After following the workup above, solid product was obtained from recrystallization in ethanol and ethyl acetate mixture to give 1.6 grams of G: yield 16.7%. mp: 239-240° C. ¹H NMR: δ 7.501 (d, 1H), 7.472 (s, 1H), 7.427 (dd, 1H), 2.194 (s, 3H, Me), 2.032 (s, 3H, Me). ¹³C NMR: 180.244, 176.047, 138.409, 135.059, 132.545, 130.484, 128.649, 128.359, 110.807, 110.281, 109.033, 107.339, 99.563, 60.479, 25.521, 14.494. HPLC: 88.05%.

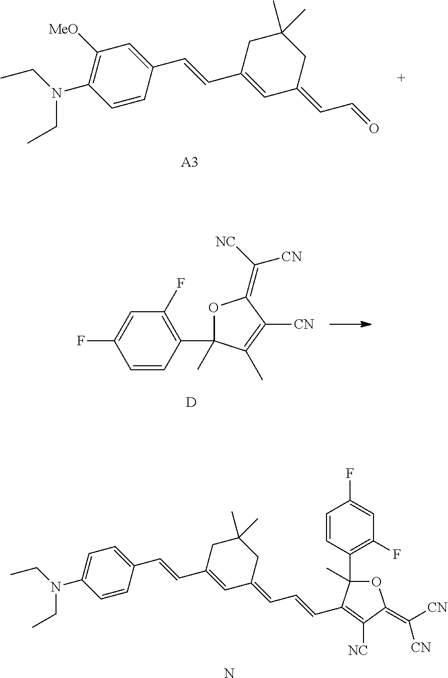

### Compound N

A3 1.11 g (0.0034 mole), D 1.22 g (0.004 mole) and piperidine were mixed in THF (30 mL)/EtOH (30 mL). This mixture was refluxed overnight. After cooled to room temperature, this mixture was worked up in ethyl acetate/hexane. The pure N was collected by crystallization from ethanol to give 1.29 g: yield 63%. HPLC: 100%.

### Compound F: 2-dicyanomethylene-3-cyano-4,5-dimethyl-5-[3′,4′-dichlorophenyl]-2,5-dihydrofuran

3-hydroxy-3-[3′,4′-dichlorophenyl]-2-butanone 15 g (0.064 mole), malononitrile 8.5 g (0.129 mole) and lithium ethoxide 3.2 ml (3.2 mmole, 1M solution in ethanol) were stirred in 80 ml of THF solution and allowed to boil under reflux conditions overnight. The solution was concentrated by removing the majority of the THF on a rotary evaporator under aspirator vacuum. The remaining residue was taken up in methylene chloride, washed with brine (2×) then DI water (2×). The organic layer was dried over anhydrous MgSO4, filtered and the solvent removed. The crude product was recrystallized from denatured alcohol to yield the targeted compound F to give 5.5 grams: yield 25.9%. mp: 226.4-228.6° C. ¹H NMR: δ 7.579 (d, 1H), 7.324 (d, 1H), 7.070 (dd, 1H), 2.254 (s, 3H, Me), 2.001 (s, 3H, Me). ¹³C NMR: 180.253, 175.015, 135.676, 134.630, 134.382, 131.982, 127.552, 124.595, 110.580, 109.966, 108.810, 105.822, 100.107, 60.573, 22.894, 22.894, 14.637. HPLC: 100%.

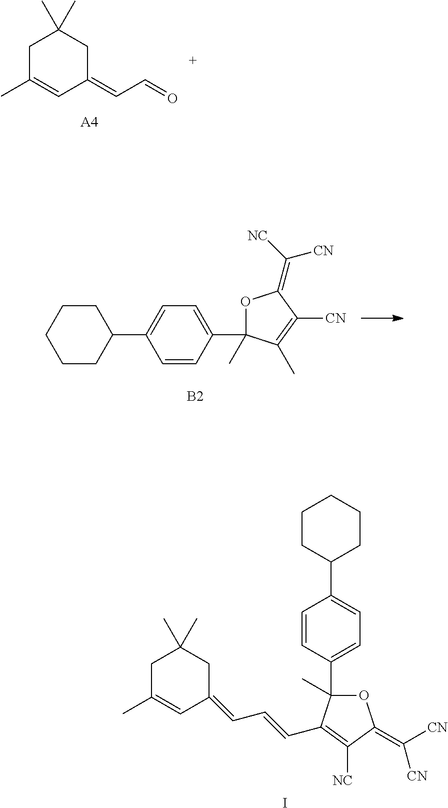

### Compound I

To a mixture of A4 0.5 g (0.003 mole) and B2 1.0 g (0.003 mol), 40 mL of THF and 10 mL of EtOH were added. While this mixture was heated at 70 to 800 C, 5 drops of anhydrous piperidine were added. This mixture was heated at this temperature for 16 hours. The reaction mixture was then cooled to room temperature and solvents were removed under reduced pressure to yield an oil which was dissolved in methylene chloride. This methylene chloride solution was washed by water and dried over anhydrous MgSO₄ and concentrated to form an oily product. The pure I was obtained through a column chromatography on silica to give 0.3 grams: yield 20%. HPLC: 100%.

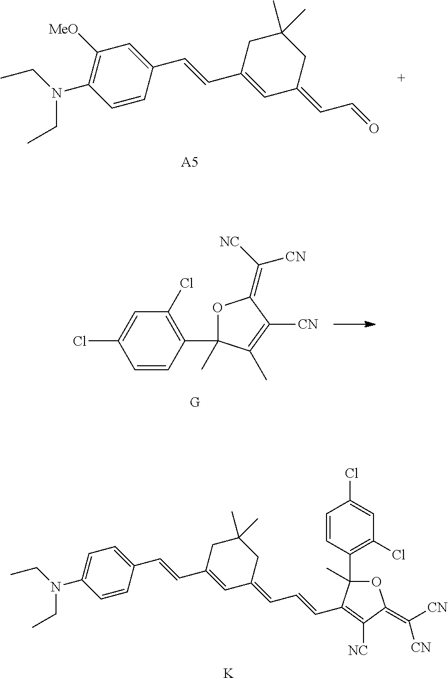

### Compound K

A5 0.45 g (0.001 mole), G 0.45 g (0.001 mole) and 3 drops of piperidine were mixed in THF (40 mL)/EtOH (20 mL). This mixture was refluxed overnight. After cooled to room temperature, this mixture was worked up in ethyl acetate/hexane to yield the desired product. The pure K was collected by crystallization from ethanol to give 0.15 g: yield 23%. HPLC: 100%.

### B. Example 2

### Label-Free Optical Biosensor Cellular Assay Characterization of Compounds

Optical biosensors primarily employ a surface-bound electromagnetic wave to characterize cellular responses. The surface-bound waves can be achieved on metallic substrates (such as gold) using either light excited surface plasmons (surface plasmon resonance, SPR) or on dielectric substrates using diffraction grating coupled waveguide mode resonances (resonance waveguide grating, RWG). For SPR, the readout is the resonance angle at which a minimal in intensity of reflected light occurs. Similarly, for RWG biosensor, the readout is the resonance angle or wavelength at which a maximum incoupling efficiency is achieved. Photonic crystal biosensor is a RWG biosensor. The resonance angle or wavelength is a function of the local refractive index at or near the sensor surface. Unlike SPR which is limited to a few of flow channels for assaying, RWG biosensors are amenable for high throughput screening (HTS) and cellular assays, due to recent advancements in instrumentation and assays. In a typical RWG, the cells are directly placed into a well of a microtiter plate in which a biosensor consisting of a material with high refractive index is embedded. Local changes in the refractive index lead to a dynamic mass redistribution (DMR) signal of live cells upon stimulation. These biosensors have been used to study diverse cellular processes including receptor biology, ligand pharmacology, and cell adhesion.

Using the RWG biosensor Corning® Epic® system, all compounds were systematically characterized for their ability to modulate hERG ion channels in live cells. The Epic® system consists of a temperature-control unit, an optical detection unit, with an on-board liquid handling unit with robotics, or an external liquid accessory system with robotics. The detection unit is centered on integrated fiber optics, and enables kinetic measures of cellular responses with a time interval of ˜7 or 15 sec. The compound solutions were introduced by using either the on-board liquid handling unit, or the external liquid accessory system; both of which use conventional pippetting system.

1. Materials and Methods

a) Cell Culture

All cell culture reagents were purchased from Invitrogen GIBCO cell culture products. HEK293, MCF7 and HT29 cells were purchased from ATCC. HEK293 cells were maintained in MEM-GlutoMax with 10% fetal bovine serum and 1% Penicillin/streptomycin according to ATCC's instructions. HT29 cells were maintained in McCoy's 5A medium with 10% fetal bovine serum and 1% Penicillin/streptomycin. MCF7 cells were maintained in ATCC-formulated Eagle's Minimum Essential Medium containing 0.01 mg/ml bovine insulin, fetal bovine serum to a final concentration of 10%. HEK hERG stable cell line (HEK-hERG) was maintained according to Sun et al. (J. Biol. Chem. 2006, 281:5877). Cells were subcultured 1-2 times per week and cell passage less than 15 was used for all experiments. All cell cultures were taken place in 5% carbon dioxide in typical cell culture incubator.

b) Compounds

Mallotoxin, flufenamic acid, difunisal, and dofetilide were purchased from Enzo Lifesciences. Dofetilide was also purchased from Fisher Scientific.

c) Label-Free Biosensor Cellular Assays

Epic® wavelength interrogation system (Corning Inc., Corning, N.Y.) was used for whole cell sensing. This system consists of a temperature-control unit, an optical detection unit, and an on-board liquid handling unit with robotics. The detection unit is centered on integrated fiber optics, and enables kinetic measures of cellular responses with a time interval of ˜15 sec.

Cells were plated in 384-well Epic® cell culture treated plate (Corning Cat#5040) 16-20 hours before assay (15000 cells/well for HEK293 and HEK-hERG cells, 30000 cells/well for HT29 cells). For both HEK-hERG and its parental HEK293 cells, each well was coated with 10 μl 5 μg/ml fibronectin. One hour before assay, cells were washed twice on a BioTek ELx405 Select washer with Hank's Balanced Salt Solution (HBSS) containing 20 mM Hepes. Cells were incubated in 40 μl/well HBSS at 28° C. inside the Epic system for one hour. For each assay, a 2-min baseline was initiated, followed by addition of 10 μl compound solutions (5×) and the cell responses were recorded continuously for one hour.

All studies were carried out at a controlled temperature (28° C.). At least two independent sets of experiments, each with at least three replicates, were performed. The assay coefficient of variation was found to be <10%. All dose-dependent responses were analyzed using non-linear regression method with the GraphPad Prism 5.

d) Rb⁺ Flux Assay

Rb⁺ flux assay was performed using HEK-hERG cells as described in previous literature (Sun, H., et al., J. Biol. Chem. 2006, 281: 5877). Briefly, 50,000 cells per well were plated in 96-well tissue culture treated plates 20 hours before assay. In the next day, cells were incubated with complete cell culture medium containing 5 mM RbCl for 3 hours at 37° C. with 5% CO₂. Then compounds (10×) diluted in HBSS were added into the cell culture medium and cells were incubated at 37° C. with 5% CO₂ for another hour. Cells were washed twice with Rb⁺ free cell culture medium and incubated with 180 μl/well of cell culture medium containing different concentration of KCl for exactly 10 minutes. The supernatant from each well was transferred to a new 96 well plate immediately. Cells were lysed with 180 μl/well 0.5% Triton100 in HBSS. The Rb⁺ concentration of each sample was determined by ICR8000 (Aurora Biomed Inc.).

e) Cell Proliferation Assays

Proliferation was measured using the CellTiter-Glo Luminescent Cell Viability Assay (Technical Bulletin #288, Promega, Madison, Wis.). When added to cells, the assay reagent produces luminescence in the presence of ATP from viable cells. Cells were plated in 96-well Corning Costar TCT (tissue culture treated) plates at a density of 10,000 cells/well and incubated for 24 h. Test samples were solubilized in dimethyl sulfoxide (DMSO) by sonication, filter sterilized and diluted with media to the desired treatment concentration. Cells were treated with 100 μl control media, or test samples, and incubated for 48 h drug exposure duration. At the end of 48 h, plates were equilibrated at room temperature for 30 min, 100 μl of the assay reagent was added to each well and cell lysis was induced on an orbital shaker for 2 min. Plates were incubated at room temperature for 10 min to stabilize the luminescence signal and results were read on an Perkin Elmer Vector 3 Microplate Reader. All plates had control wells containing medium without cells to obtain a value for background luminescence. Data are expressed as for three replications.

f) Automated Patch Clamp Recording Using IonWorks

CHO-K1 cells stably expressing hERG channel (CHO-hERG) were cultured in T175 flask till about 70% confluent. Cells were washed twice with PBS, then 2.5 ml 0.25% Trypsin/EDTA was mixed with 2.5 ml PBS and added to the T175 flask. Cells were incubated about 2 minutes with the diluted Trypsin/EDTA solution at 37° C., then were continuously incubated about 3 minutes at room temperature. 20 ml fresh medium were added to suspend the cells and transfer to a 50 ml tube. Cells were centrifuged down at 750 rpm for 5 minutes. The extra medium was removed and cells were resuspended in 6 ml External Buffer (137 mM NaCl, 4 mM KCl, 1.8 mM CaCl₂, 1 mM MgCl₂, 10 mM HEPES, 10 mM glucose, pH 7.4). Then cells were centrifuged again at 450 rpm for another 5 minutes. Finally cells were resuspended in 4 ml the External Buffer, cell number was counted using a hemacytometer. Cell suspension was diluted to 2.5×10⁶ cells/ml with the External Buffer. 4 ml of the resuspended cells were added to the cell reservoir in IonWorks. The Internal solution used contains: 40 mM KCl, 100 mM K-Gluconate, 3.2 mM MgCl₂, 2 mM CaCl₂, 5 mM HEPES, pH 7.25 (adjusted with KOH). 5 mg Amphotericin B from 200 ul DMSO stock was added to 65 ml the Internal solution and mixed well to achieve electrical access to the interior of cells on the patch plate.

Compounds were prepared from 10 mM DMSO stock and diluted in the External Buffer to make 3× compound solution. 60 μl/well of the 3× compound solution was transferred to a 384 well plate in Row A, B, C and D. PD11857 (Sigma-Aldrich), a reported hERG channel activator was used as activator positive control (final concentration 30 and 50 μM). hERG blocker dofetilide was used as blocker positive control (final concentration 100 nM). The final DMSO concentration in the IonWorks Quattro PatchPlate PPC plate (Cat#9000-0902, Molecular Devices) was 0.5%. Final compound concentration was 50 μM. Each compound was added to four wells of one PPC plate.

hERG currents were recorded on IonWorks Quattro (Molecular Devices). To record hERG current, the cells were clamped at −80 mV initially, then followed by a 5-s depolarization at +40 mV to activate the channels. Tail currents were measured during an ensuing return to −35 mV. Data analysis were done using IonWorks Quattro® System Software version 2.0.4.4. Data from wells with seal resistance less than 50 MΩ or hERG tail currents less than 0.1 nA were filtered out. Activator hits were selected if hERG tail currents ratio (post/pre-compound) is greater than mean+2SD (standard deviation) of the average DMSO control. Inhibitor hits were selected if hERG tail currents ratio (post/pre-compound) is less than mean-2SD of the average DMSO control.

2. Results

The human ether-à-go-go related gene (hERG) product encodes for the pore-forming subunit of the rapid component of the delayed rectifier K⁺ channel that mediates repolarization of cardiac action potential. hERG is a voltage gated ion channel, and involved in regulating the movement of potassium ions across the cell plasma membrane. Since hERG is a quite large ion channel, and at least in many cancerous cells it can co-exist with several other signaling molecules including integrins and/or receptor tyrosine kinases to form a large signaling complex. There is evidence showing that hERG channel is involved in cell signaling. Furthermore, the recent discovery of several hERG activators, although via different mechanisms, can cause the activation of hERG channels at or near physiological conditions. Label-free RWG biosensor cellular assays were developed to directly assaying the activation and its subsequent signaling of hERG channels without applying voltages to the cells. A method was also developed to validate the ability of any compounds to modulate, particularly activate, the hERG ion channels. Generally, the disclosed methods to characterize modulators acting directly through hERG ion channel, or indirectly via hERG-associated signaling complexes are related to label-free biosensor cellular assays. Disclosed is the use of three types of cells: a cancerous cell line endogenously expressing hERG ion channels, a native cell line without endogenous hERG channels and its engineered cell line overexpressing hERG channels, for characterizing hERG modulators. Also disclosed is the use of mallotoxin or other hERG activator as a readout to further confirm the modes of action of hERG modulators. In addition, disclosed are methods that utilize additional assays, such as an ion flux assay, such as an Rb+ assay, membrane potential fluorescence assays, or patch clamping assays

Native cell lines endogenously expressing hERG channels include, but are not limited to, leukemia cell line HL60, gastric cancer cell line SGC7901 and MGC803, neuroblastoma cell line SH-SY5Y, mammary carcinoma cell line MCF-7, and human colon carcinoma cell HT-29, HCT8, and HCT116. Native cell lines without hERG include, but are not limited to, human embryonic kidney cell line HEK-293, and Chinese Ovary hamster cell line CHO-K1. Engineered cell lines overexpressing hERG include, but are not limited to, HEK-hERG and CHO-hERG cells. Cardiovascular or neuronal cells including primary cells having endogenous hERG channels can also be used.

hERG activators include, but are not limited to, mallotoxin, RPR260243, NS1643, NS3623, PD-118057, PD-307243, and A-935142.

In a specific approach, any compound can be profiled in three different types of cells, HT29, HEK-hERG, and HEK-293 cells, in both agonism and antagonism modes. The human colon cancerous cell line HT-29 is known to endogenously express hERG channels. HEK293 is a native cell line without endogenously expressed hERG channels, while HEK-hERG is an engineered HEK293 cell having stably expressed hERG channels. Mallotoxin was also chosen as the label-free biosensor hERG activator. Mallotoxin is a hERG activator to activate hERG channels at or near physiological conditions (i.e., 1×HBSS buffered conditions).

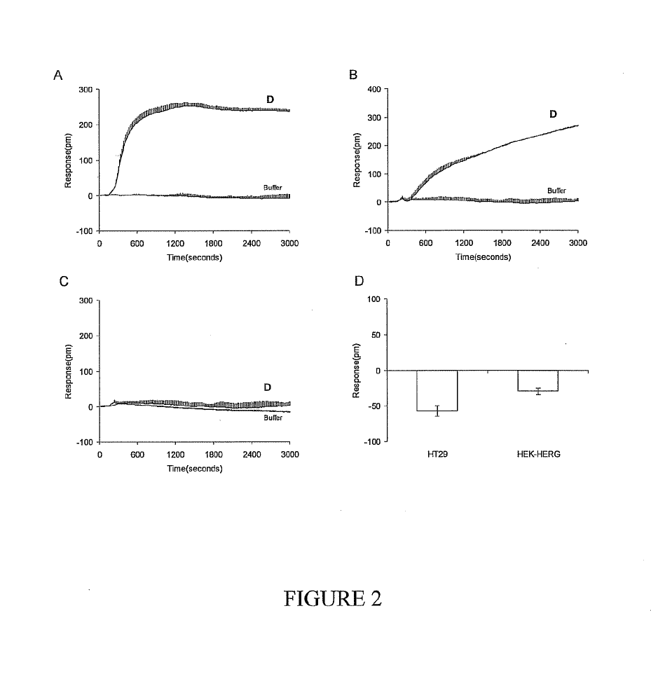

As shown in FIGS. 1 and 2, both compounds E and D led to robust DMR signals in both HT29 and HEK-hERG cells but not HEK-293 cells. In addition, both compounds, each at 10 micromolar, gave rise to an attenuated signal of the mallotoxin responses in both HT29 and HEK-hERG cells, using the typical antagonism assays. Due to different expression level in hERG channels as well as the organization of hERG channel signaling complexes, the modulation of the mallotoxin DMR signals in HT29 and HEK-hERG by different hERG modulators is expected to differ greatly. These results indicate that both E and D act as hERG activators. Similar results were also observed for the compounds P, M, Q, U, R, B, V, T, W, G, L, S, N, O, F, J, H, I, C, A, K (data not shown).

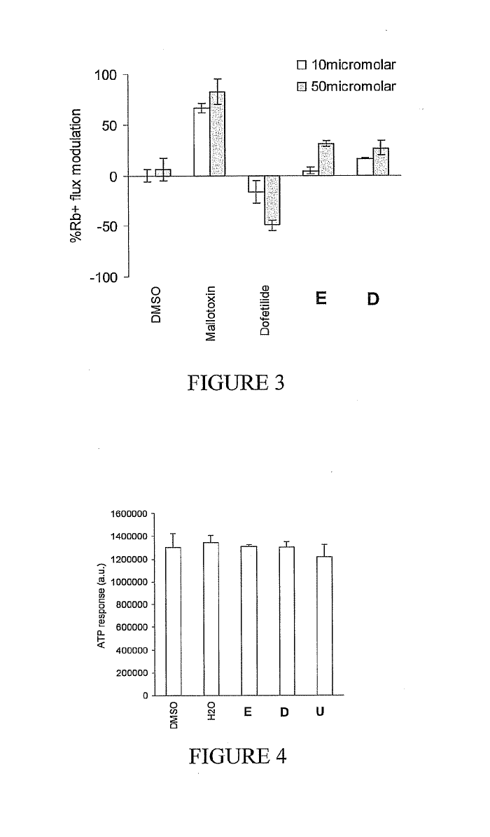

To confirm these findings, Rb+ flux assays were used to characterize both compounds. As shown in FIG. 3, mallotoxin at either 10 or 50 μM led to a significant increase in Rb⁺ signaling when the cells were maintained in a buffered solution containing 5 mM KCl. In contrast, the known hERG blocker dofetilide caused the suppression of Rb⁺ signal in HEK-hERG cells. Both compounds E and D (at 10 or 50 micromolar) also led to a small but detectable increase in Rb⁺ flux. These results showed that both E and D act as hERG ion flux activators.

Cell proliferation assays using both HT29 and MCF7 showed that all three compounds E, D and U did not lead to cell apoptosis or any alteration in cell proliferation rate (FIG. 4 and data not shown).

Using similar methods, the non-steroidal anti-inflammatory drug flufenamic acid was found to be a hERG activator. Flufenamic at 10 micromolar lead to a robust DMR signals in both HT-29 and HEK-hERG cells (FIG. 5A and FIG. 5B, respectively), but not HEK293 cells (FIG. 5C). Flufenamic acid selectively attenuated the mallotoxin DMR signal in HT-29 cells, but not HEK-hERG cells (FIG. 5D). Although the DMR signals of flufenamic acid in both hERG-expressing cell lines are quite different from the corresponding DMR signals of mallotoxin, these results indicate that flufenamic acid acts as a weak hERG activator. Follow up studies with Rb⁺ flux assays showed that flufenamic acid indeed caused a small and dose-dependent increase in Rb⁺ signal when HEK-hERG was assayed in 5 mM KCl (data not shown). The patch clamping recording also suggested that flufenamic acid is a hERG current activator, as shown in FIG. 5E. The Curve 510 showed the hERG currents before flufenamic acid addition, while the curve 520 showed the hERG currents after flufenamic acid addition. The tail current, as indicated in FIG. 5E (b), is potentiated by flufenamic acid. Similarly, niflumic acid was also found to be a label-free biosensor hERG activator, a hERG current activator, and a hERG activator (data not shown). However, diflunsial was found to be a label-free biosensor hERG activator, a hERG pathway activator, but not a hERG current activator (FIG. 6). To record hERG current, the cells were clamped at −80 mV initially, then followed by a 5-s depolarization at +40 mV to activate the channels (phase a in FIG. 5D). Tail currents were measured during an ensuing return to −35 mV for 2 seconds (phase b in FIG. 5D). Final holding potential at −70 mV (phase c in FIG. 5D). It has not been reported in literature that niflumic acid is a hERG activator.

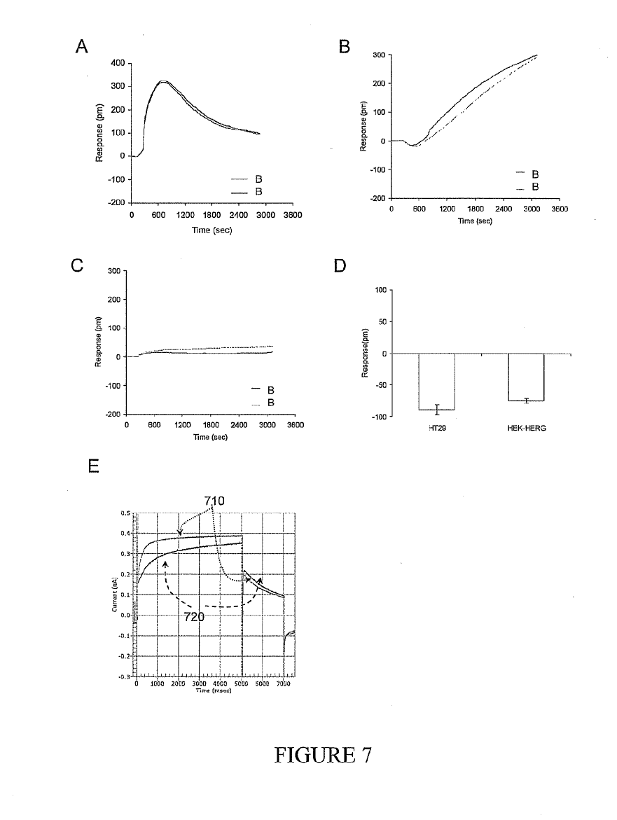

Interestingly, B was found to be a potent label-free biosensor hERG activator, as evidenced by its robust DMR signals in both HT-29 and HEK-hERG cells (FIG. 7a and b, respectively), but not in HEK293 cells (FIG. 7c). B caused desensitization of both cells to the subsequent mallotoxin stimulation (FIG. 7d). B is also a weak hERG current activator, as evidenced by the potentiated tail current (the phase b) of hERG channel in CHO-hERG cells (FIG. 7E). W led to similar profiles as B (data not shown).

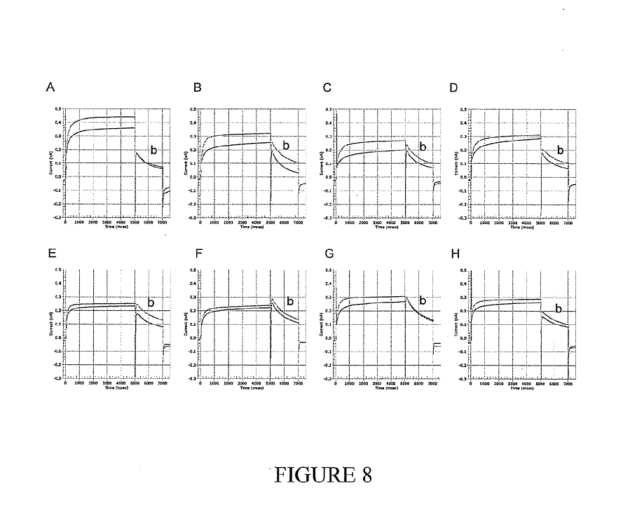

Conversely, although A, C, D, F, H, I, J and U were found to be label-free biosensor hERG activator, these compounds appear have minimal impact on the hERG current in CHO-hERG cells, as recorded using the automated patch clamping (FIG. 8). These results suggest that these compounds act as a label-free biosensor hERG activator and a hERG pathway activator, and are possibly extremely weak, or not at all, hERG ion channel activators. Compounds E, G, K, L, M, N, O, P, Q, S, T and V were not tested using the automated patch clamp method.

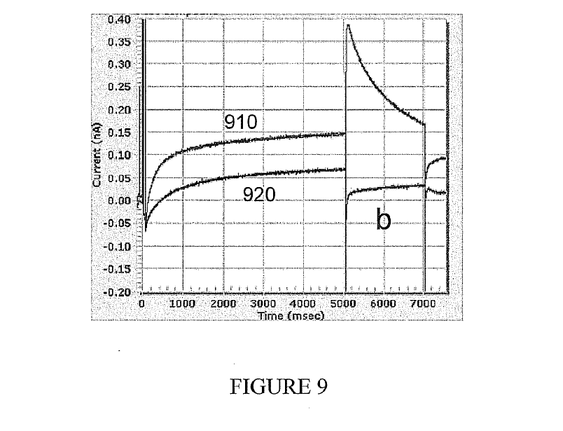

As a control, the classical hERG blocker dofetilide at 100 nM completely inhibited the tail current of hERG channel in CHO-hERG cells (FIG. 9).

### III. SEQUENCES

The sequence of hERG protein is disclosed in SEQ ID NO: NP_{—}000229. The sequence of hERG gene is disclosed in SEQ ID NO: NM_{—}000238.

### REFERENCES

U.S. Ser. No. 12/623,693. Fang, Y., Ferrie, A.M., Lahiri, J., and Tran, E. “Methods for Characterizing Molecules”, Filed Nov. 23, 2009
U.S. Ser. No. 12/623,708. Fang, Y., Ferrie, A.M., Lahiri, J., and Tran, E. “Methods of creating an index”, filed Nov. 23, 2009.
U.S. Pat. No. 6,393,190B1 He, M., Leslie, T. M. “Chromophores for polymeric thin films and optical waveguides and devices comprising the same”
Cavalli, A.; Poluzzi, E.; De Ponti, F.; Recanatini, M., Toward a pharmacophore for drugs inducing the long QT syndrome: insights from a CoMFA study of hERG K(+) channel blockers. J Med Chem 2002, 45, (18), 3844-53.
Bridgland-Taylor, M. H.; Hargreaves, A. C; Easter, A.; Orme, A.; Henthorn, D.C.; Ding, M.; Davis, A. M.; Small, B. G.; Heapy, C. G.; Abi-Gerges, N.; Persson, F.; Jacobson, L; Sullivan, M.; Albertson, N.; Hammond, T. G.; Sullivan, E.; Valentin, J. P.; Pollard, C. E., Optimisation and validation of a medium-throughput electrophysiology-based hERG assay using IonWorks HT. J Pharmacol Toxicol Methods 2006, 54, (2), 189-99
Dubin, A. E.; Nasser, N.; Rohrbacher, J.; Hermans, A. N.; Marrannes, R.; Grantham, C; Van Rossem, K.; Cik, M.; Chaplan, S. R.; Gallacher, D.; Xu, J.; Guia, A.; Byrne, N. G.; Mathes, C, Identifying modulators of hERG channel activity using the PatchXpress planar patch clamp. J Biomol Screen 2005, 10, (2), 168-81
Warmke J. W., et al. (A family of potassium channel genes related to eag in Drosophila and mammals. PNAS. 1994. 91 (8):3438-3442 incorporated by reference

1. A method of modulating a hERG ion channel, comprising administering one or more compounds having the formula: 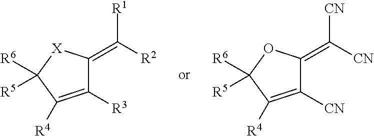
wherein X is O or S;
wherein R¹, R² and R³ independently are CN or an electron withdrawing group;
wherein R⁴ is H, C₁-C₁₀ alkyl, alkenyl, alkynyl, fully conjugated chromophore with electronic donating-bridge-accepting structure, donating-accepting or bridge-accepting structures or 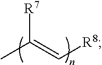
wherein n is 1-6;
wherein R⁷ is H or C₁-C₆ alkyl;
wherein R⁸ is H, C₁-C₃ alkyl, alkenyl, alkynyl, 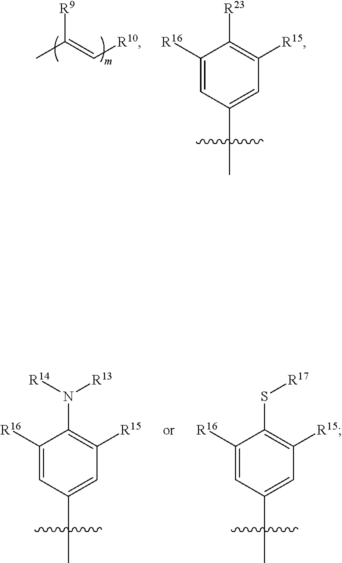
wherein m is 0-1;
wherein R⁹ is H or C₁-C₆ alkyl, wherein R⁷ and R⁹ are optionally cyclized to form a 4- to 8-membered ring;
wherein R¹³ and R¹⁴ are independently H, C₁-C₆ alkyl, alkenyl or alkynyl;
wherein R¹⁵ and R¹⁶ are independently H, C₁-C₃ alkyl, alkenyl, alkynyl or alkoxy;
wherein R¹⁷ is H, C₁-C₆ alkyl, alkenyl or alkynyl;
wherein R²³ is amino, alkylamino, dialkylamino, dialkylanilino, 1-piperidino, 1-piperazino, 1-pyrrolidino, acylamino, hydroxyl, thiolo, alkylthio, arylthio, alkoxy, aryloxy, acyloxy, alkyl, vinyl, or 1,2,3,4-tetrahydroquinolinyl;
wherein R¹⁰ is H, C₁-C₃ alkyl, alkenyl, alkynyl, 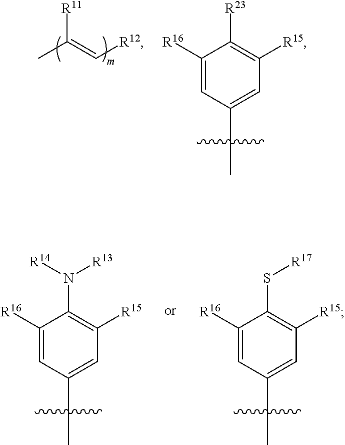
wherein o is 0-6;
wherein R¹¹ is H, or C₁-C₆ alkyl, wherein R⁹ and R¹¹ are optionally cyclized to form a 4- to 8-membered ring;
wherein R¹² is H, C₁-C₃ alkyl, alkenyl, alkynyl, 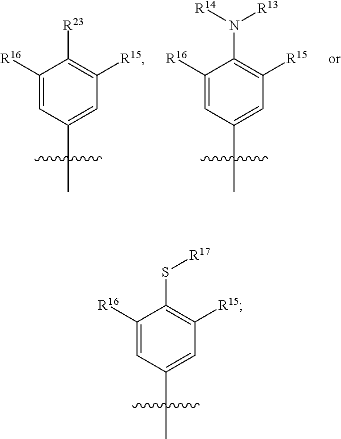
wherein R⁵ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 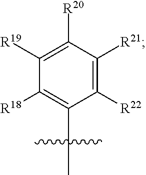
R¹⁸, R¹⁹, R²⁰, R²¹ and R²² are independently H, halogen, Cl, F, C₁-C₆ alkyl, alkenyl, alkynyl, aryl, C₃-C₈ cycloalkyl;
wherein R⁶ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 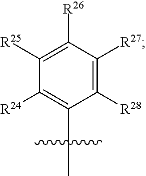
R²⁴, R²⁵, R²⁶, R²⁷ and R²⁸ are independently H, halogen, Cl, F, C₁-C₆ alkyl, alkenyl, alkynyl, aryl, C₃-C₈ cycloalkyl;
wherein R⁵ and R⁶ are optionally cyclized; and
wherein the compound is a hERG modulator.

2. The method of claim 1, having the formula: 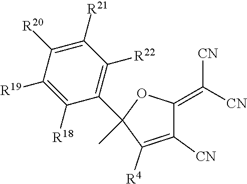
wherein R⁴ is H, C₁-C₁₀ alkyl, alkenyl, alkynyl, fully conjugated chromophore with electronic donating-bridge-accepting structure, donating-accepting or bridge-accepting structures or 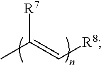
wherein n is 1-6;
wherein R⁷ is H or C₁-C₆ alkyl;
wherein R⁸ is H, C₁-C₃ alkyl, alkenyl, alkynyl, 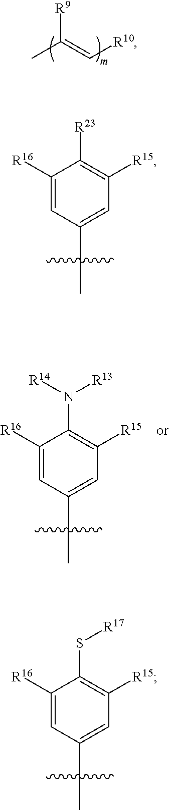
wherein m is 0-1;
wherein R⁹ is H or C₁-C₆ alkyl, wherein R⁷ and R⁹ are optionally cyclized to form a 4- to 8-membered ring;
wherein R¹³ and R¹⁴ are independently H, C₁-C₆ alkyl, alkenyl or alkynyl;
wherein R¹⁵ and R¹⁶ are independently H, C₁-C₃ alkyl, alkenyl, alkynyl or alkoxy;
wherein R¹⁷ is H, C₁-C₆ alkyl, alkenyl or alkynyl;
wherein R²³ is amino, alkylamino, dialkylamino, dialkylanilino, 1-piperidino, 1-piperazino, 1-pyrrolidino, acylamino, hydroxyl, thiolo, alkylthio, arylthio, alkoxy, aryloxy, acyloxy, alkyl, vinyl, or 1,2,3,4-tetrahydroquinolinyl;
wherein R¹⁰ is H, C₁-C₃ alkyl, alkenyl, alkynyl, 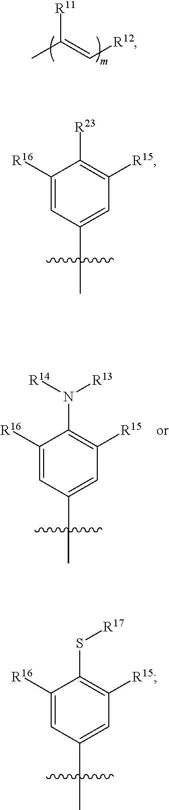
wherein o is 0-6;
wherein R¹¹ is H, or C₁-C₆ alkyl, wherein R⁹ and R¹¹ are optionally cyclized to form a 4- to 8-membered ring;
wherein R¹² is H, C₁-C₃ alkyl, alkenyl, alkynyl, 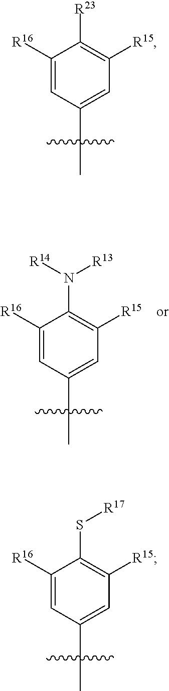
wherein R⁵ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 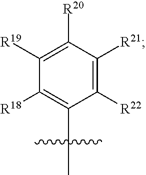
wherein R¹⁸, R¹⁹, R²⁰, R²¹ and R²² are independently H, halogen, Cl, F, C₁-C₆ alkyl, alkenyl, alkynyl, aryl, C₃-C₈ cycloalkyl;
wherein R⁶ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 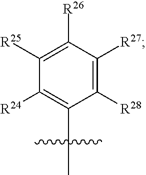
wherein R²⁴, R²⁵, R²⁶, R²⁷ and R²⁸ are independently H, halogen, Cl, F, C₁-C₆ alkyl, alkenyl, alkynyl, aryl, C₃-C₈ cycloalkyl.

3. The method of claim 2, wherein R⁴ is

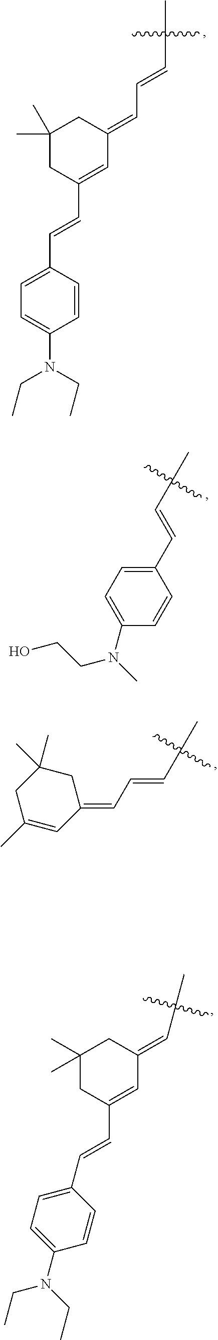

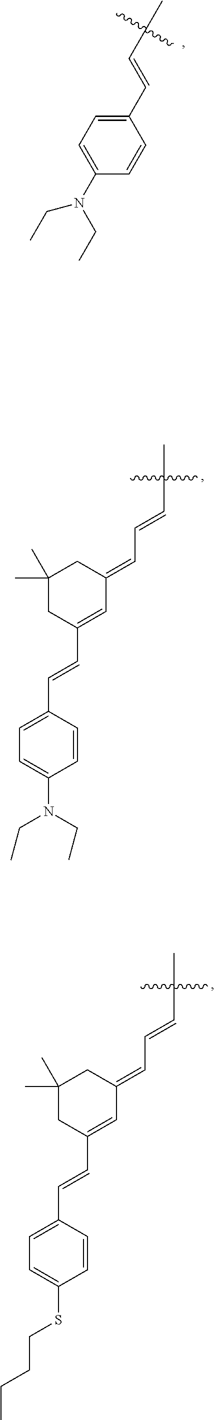

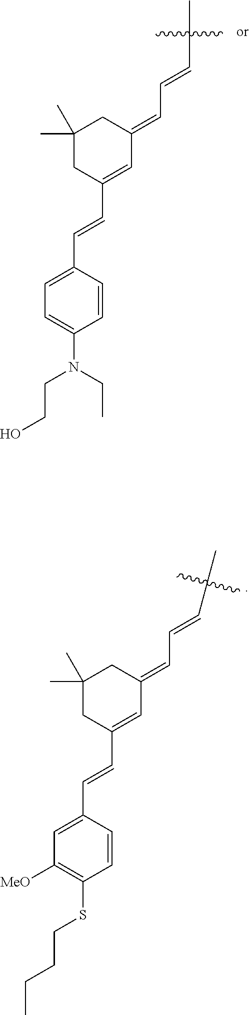

4. The method of claim 1, having the formula 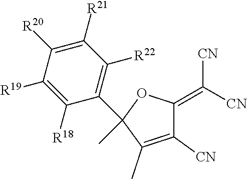
wherein R¹⁸, R¹⁹, R²⁰, R²¹ and R²² are independently H, halogen, Cl, F, C₁-C₆ alkyl, alkenyl, alkynyl, aryl, or C₃-C₈ cycloalkyl.

5. The method of claim 1, having the formula 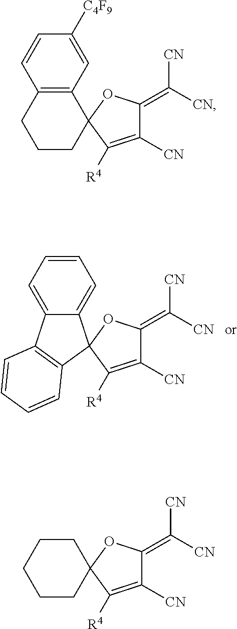
wherein R⁴ is H, C₁-C₁₀ alkyl, alkenyl, alkynyl, fully conjugated chromophore with electronic donating-bridge-accepting structure, donating-accepting or bridge-accepting structures or 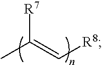
wherein n is 1-6;
wherein R⁷ is H or C₁-C₆ alkyl;
wherein R⁸ is H, C₁-C₃ alkyl, alkenyl, alkynyl, 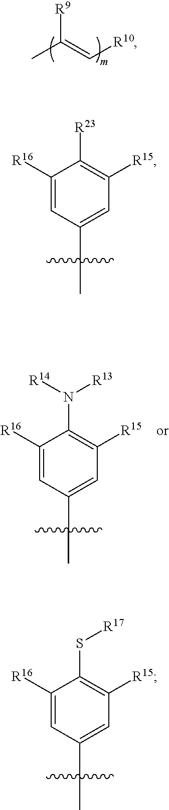
wherein m is 0-1;
wherein R⁹ is H or C₁-C₆ alkyl, wherein R⁷ and R⁹ are optionally cyclized to form a 4- to 8-membered ring;
wherein R¹³ and R¹⁴ are independently H, C₁-C₆ alkyl, alkenyl or alkynyl;
wherein R¹⁵ and R¹⁶ are independently H, C₁-C₃ alkyl, alkenyl, alkynyl or alkoxy;
wherein R¹⁷ is H, C₁-C₆ alkyl, alkenyl or alkynyl;
wherein R²³ is amino, alkylamino, dialkylamino, dialkylanilino, 1-piperidino, 1-piperazino, 1-pyrrolidino, acylamino, hydroxyl, thiolo, alkylthio, arylthio, alkoxy, aryloxy, acyloxy, alkyl, vinyl, or 1,2,3,4-tetrahydroquinolinyl;
wherein R¹⁰ is H, C₁-C₃ alkyl, alkenyl, alkynyl, 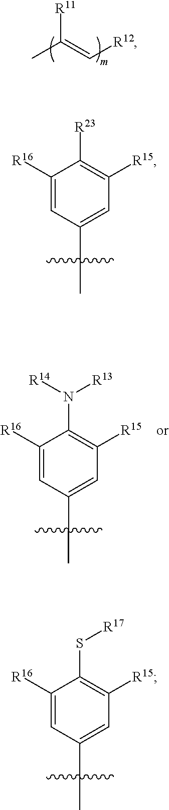
wherein o is 0-6;
wherein R¹¹ is H, or C₁-C₆ alkyl, wherein R⁹ and R¹¹ are optionally cyclized to form a 4- to 8-membered ring;
wherein R¹² is H, C₁-C₃ alkyl, alkenyl, alkynyl, 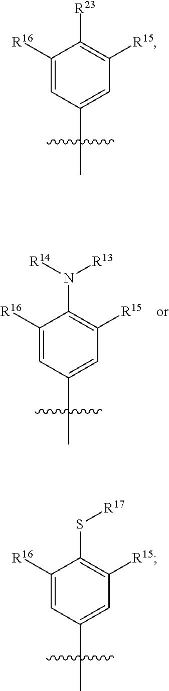
wherein R⁵ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 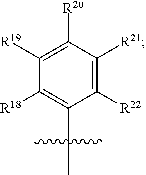
wherein R¹⁸, R¹⁹, R²⁰, R²¹ and R²² are independently H, halogen, Cl, F, C₁-C₆ alkyl, alkenyl, alkynyl, aryl, C₃-C₈ cycloalkyl;
wherein R⁶ is H, C₁-C₆ alkyl, alkenyl, alkynyl, aryl, hetero aryl, phenyl, alkylaryl, carbocyclyl, heterocyclyl, cyclohexyl, or —(CH₂)n-O—(CH₂)n, wherein n is 1-10 or 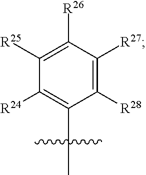
wherein R²⁴, R²⁵, R²⁶, R²⁷ and R²⁸ are independently H, halogen, Cl, F, C₁-C₆ alkyl, alkenyl, alkynyl, aryl, C₃-C₈ cycloalkyl.

6. The method of claim 5, wherein R⁴ is:

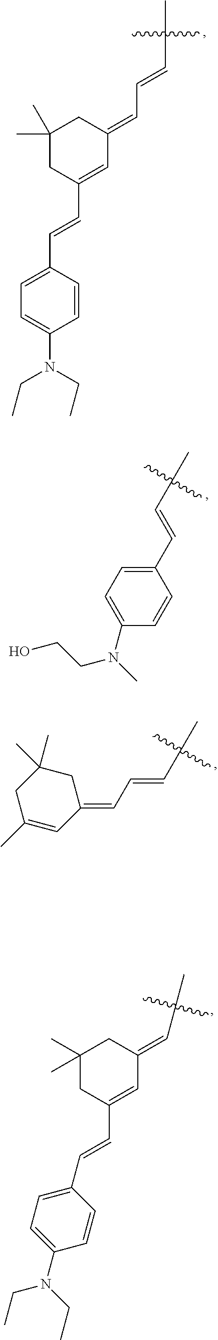

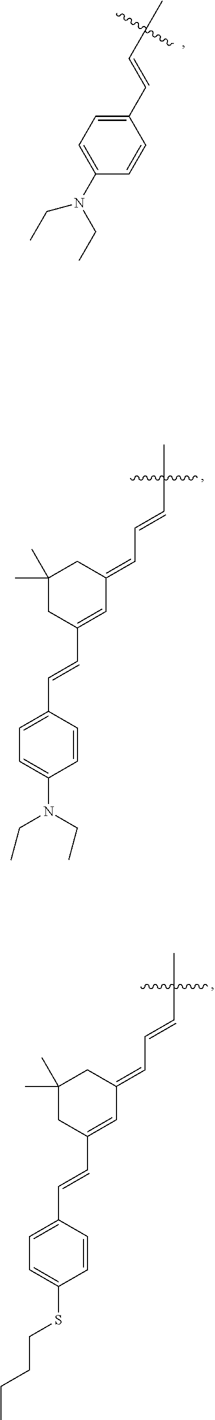

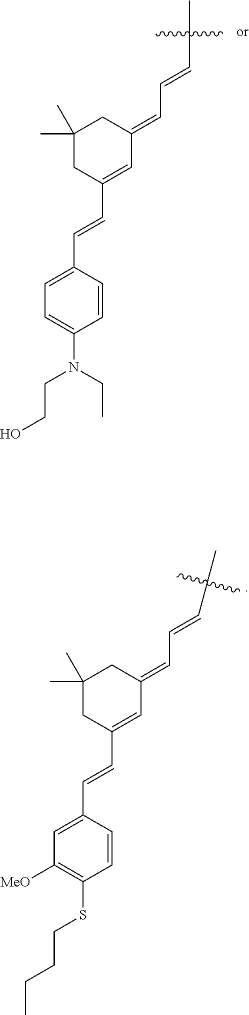

7. The method of claim 1, wherein the compound is chosen from:

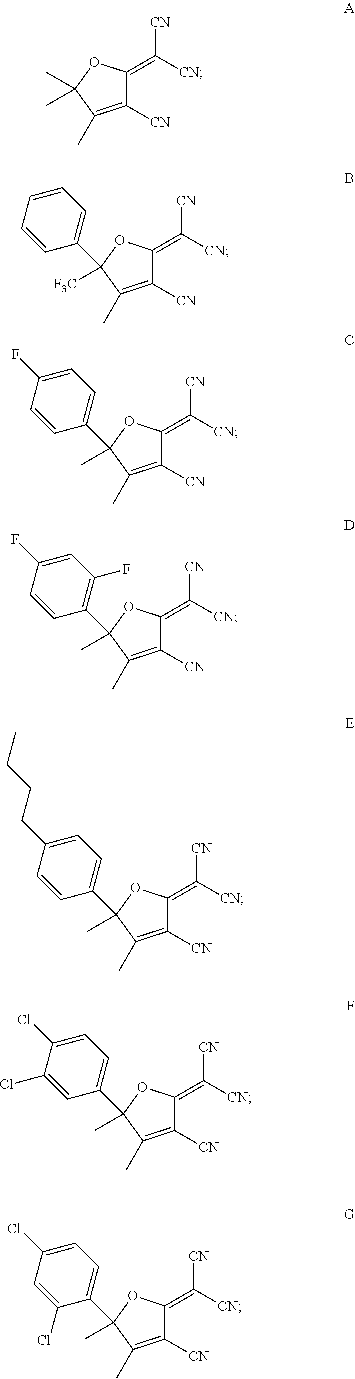

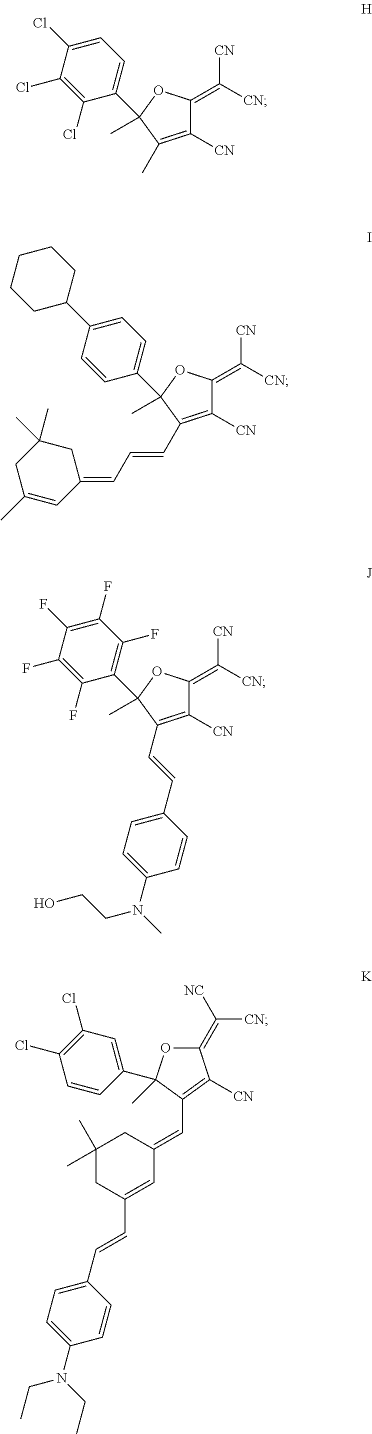

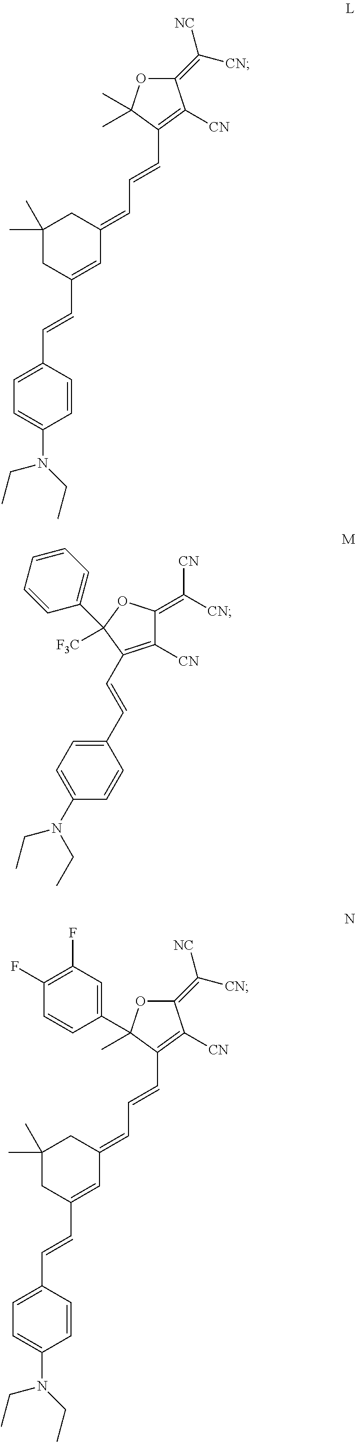

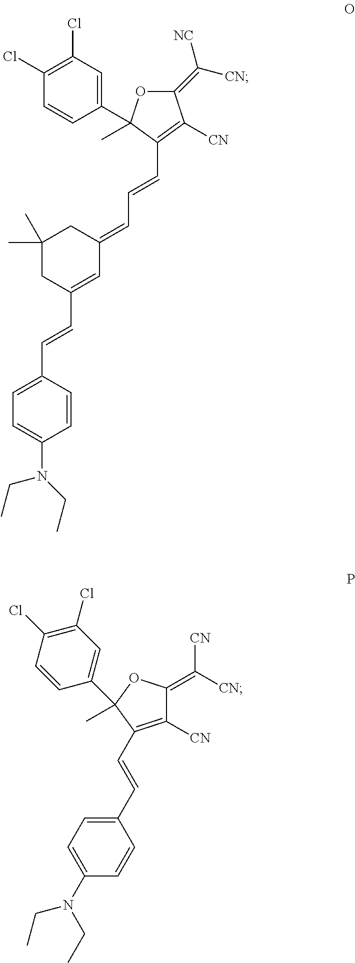

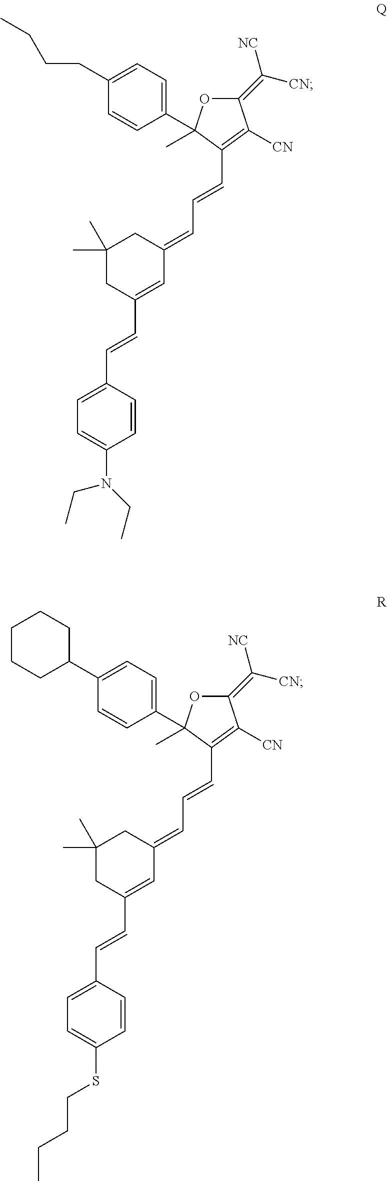

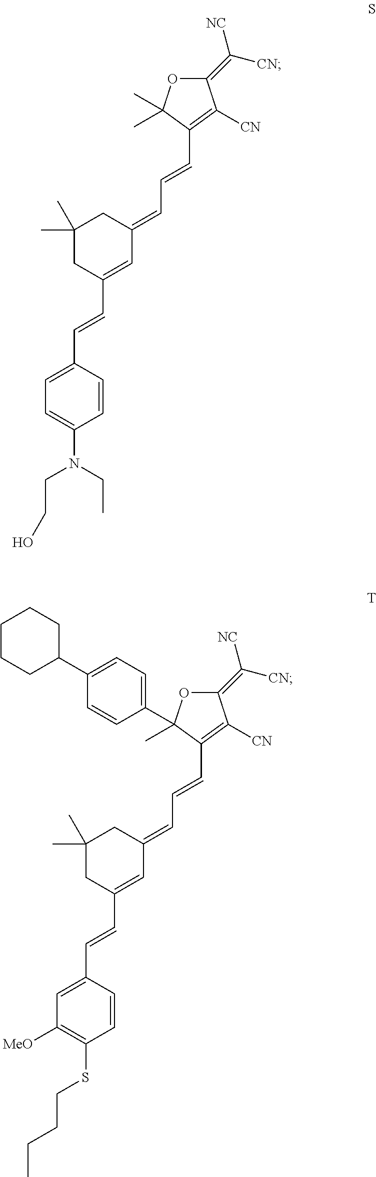

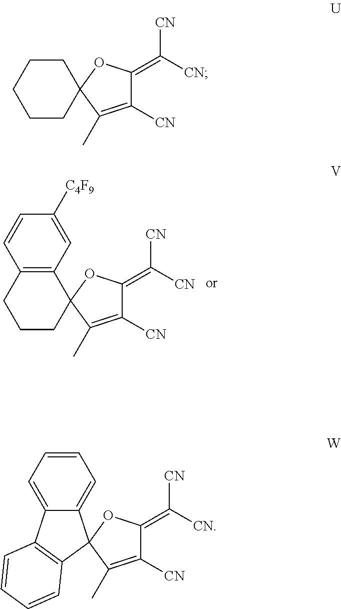

8. The method of claim 1, wherein the compound administered to a subject is a hERG modulator.

9. The method of claim 8, wherein the hERG modulator is a hERG activator.

10. The method of claim 9, wherein the subject is in need of a hERG activator to treat or prevent a disease.

11. The method of claim 10, wherein the disease is drug-induced acquired LQTS.

12. The method of claim 9, wherein the hERG activator is co-administrated with a drug having a side effect as a hERG blocker, in order to improve the safety profile of the drug.

13. The method of claim 8, wherein the hERG modulator is a hERG pathway blocker, when the subject is in need of a hERG pathway blocker to treat or prevent disease.

14. The method of claim 13, wherein the disease is leukemia, colon cancer, gastric cancer, breast cancer, or lung cancer.

15. The method of claim 10, further comprising the step of assaying the presence of the disease.

16. A method of assaying for the presence of a disease comprising assaying for the disease in a subject having been treated as in claim 10.

17. A method of modulating a hERG ion channel comprising incubating a cell comprising a hERG ion channel with a hERG modulator, wherein the modulator is a hERG pathway activator.

18. The method of claim 15, wherein the hERG pathway activator comprises a compound selected from E, P, M, D, Q, U, R, V, T, G, L, S, N, O, F, J, H, I, C, A, K, or diflunisal.

19. A method of modulating a hERG ion channel comprising incubating a cell comprising a hERG ion channel with a hERG modulator, wherein the modulator is a hERG ion channel activator.

20. The method of claim 18, wherein the hERG ion channel activator comprises a compound selected from B, W, flufenamic acid, or niflumic acid.

21.-30. (canceled)
